(19) United States
(12) Patent Application Publication
(10) Pub. No.: US 2026/0219285 A1
(43) Pub. Date: Jul. 30, 2026

(54) USE OF URINE BIOMARKERS FOR DETERMINING THE RISK OF SUBJECTS HAVING SUBCLINICAL ACIDOSIS

(71) Applicants: AARHUS UNIVERSITET, Aarhus C (DK); REGION MIDTJYLLAND, Viborg (DK)

(72) Inventors: Peder Matzen BERG, Aarhus C (DK); Mads Vaarby SØRENSEN, Aarhus C (DK); Jens Georg LEIPZIGER, Aarhus C (DK); Henrik BIRN, Aarhus C (DK); Niels Henrik BUUS, Aarhus C (DK); Samuel Levi Svinth Clement SVENDSEN, Aarhus C (DK)

(21) Appl. No.: 19/145,636

(22) PCT Filed: Jan. 31, 2024
(86) PCT No.: PCT/EP2024/052371

§ 371 Date: Jul. 3, 2025

(30) Foreign Application Priority Data
Jan. 31, 2023 (EP) 23154095.6

(51) Int. Cl. G01N 33/70; G01N 33/84

(52) U.S. Cl. CPC G01N 33/70; G01N 33/84; G01N 2800/042; G01N 2800/50; G01N 2800/52; G01N 2800/60

(57) ABSTRACT

Herein is disclosed a method for determining the risk of a subject suffering from chronic kidney disease (CKD) having subclinical acidosis and/or acid retention, the method comprising a) measuring or sensing in a urine sample from a subject, the level of at least two biomarkers b) determining a score based on a relationship between the levels of the at least two biomarkers by comparing to a reference level; and c) classifying the score as a positive or negative score; wherein said subject is a) at risk of having subclinical acidosis and/or acid retention, if said score is negative; and b) not at risk of having subclinial acidosis and/or acid retention, if said score is positive.

### TECHNICAL FIELD OF THE INVENTION

The present invention relates to a method for determining the risk of a subject suffering from chronic kidney disease (CKD) having subclinical acidosis (SA), the method comprising a) measuring or sensing in a urine sample from a subject, the level of at least two biomarkers selected from the group consisting of NH₄⁺, TA, TB, pH, BB and HCO₃⁻; b) determining a score based on a relationship between the levels of the at least two biomarkers by comparing to a reference level; and c) classifying the score as a positive or negative score; wherein said subject is a) at risk of having subclinical acidosis, if said score is negative; and b) not at risk of having subclinial acidosis, if said score is positive. In particular, the invention relates to a method for monitoring the development of SA in a subject suffering from CKD. Additionally, the invention relates to a method for determining the effect of a treatment protocol against SA for a subject suffering from CKD, and to the use of urine sample levels of at least two biomarkers for determining the risk of a subject of having subclinical acidosis and to determine and/or predict serious clinical event-free survival of said subject.

### BACKGROUND OF THE INVENTION

### 1.1. Chronic Kidney Disease (CKD): An Acid Retaining Disease.

CKD comprises a heterogeneous group of conditions that lead to a gradual loss of kidney function. With time, CKD progresses to end-stage renal disease, requiring dialysis or a kidney transplant. CKD has a high prevalence affecting 8-16% of the world's population. With progressive loss of function, the kidneys fail in their ability to remove waste products, maintain their endocrine functions, and excrete excess amounts of salts, water, and acid.

Our metabolism and diet generate a sizeable amount of non-volatile acids that need to be excreted with the urine. Since CKD patients have a significantly decreased capacity to excrete acid equivalents, they have an increased risk of accumulating acid and thus develop metabolic acidosis. Chronic metabolic acidosis in CKD patients poses an additional strain on the kidneys and becomes a driver of further organ damage and eventually terminal renal failure. Comorbidities of systemic acidosis are osteopenia, systemic inflammation and protein wasting. Oral treatment with bicarbonate can partially normalize the acidosis and has been shown to slow the progression of CKD. Current guidelines advise treating acidotic CKD patients, characterized by a plasma HCO₃⁻ of <22 mM with peroral bicarbonate supplementation.

Unfortunately, many CKD patients develop acid-mediated organ injury despite normal blood acid/base parameters. Identifying individual patients with an unobvious acid overload, which is termed subclinical acidosis (SA), preclinical acidosis or eubicarbonatemic acidosis is currently not approached in clinical practice.

### 1.2. The Potential Causes of Renal Acid Retention in CKD.

CKD is characterized by a progressive loss of glomerular filtration and functioning nephrons. Since our normal diets impose a net acid load it necessitates a positive net acid excretion in the urine. Net urinary acid excretion is obtained by effective tubular reabsorption of base (HCO₃⁻) and concurrent secretion of acid equivalents. The acid equivalents in mammalian urine are to a large degree hidden in buffers with only a minute fraction found as free protons. This means that in order to excrete large quantities of acid equivalents the tubular fluid must contain both the buffers and H⁺ to be buffered.

The key buffer system used for the elimination of acid is ammonia/ammonium. De novo synthesized NH₄⁺/NH₃ is excreted into the tubular fluid increasing the urinary buffer capacity. Concurrently, ammoniagenesis results in a net gain of base (HCO₃⁻) to the blood compartment. The proximal tubule (PT) is the site of renal ammoniagenesis and the process adapts to systemic acid accumulation. A hallmark observation in CKD patient cohorts is that urinary NH₄⁺ excretion is reduced when compared with healthy control urines.

A second urine H⁺ buffer system comprises the titratable buffers. These are filtrated complex anions (e.g. phosphates, sulfates, creatinine, citrate, and urate) derived from systemic metabolism and oral intake. The excretion of these buffers is less adaptable to acid loading.

The degree of protonation of the buffers is determined by the pH of the tubular fluid, which is a function of tubular H⁺ secretion. In the PT and the thick ascending limb of the loop of Henle (TAL), H⁺ secretion is coupled to Na⁺ and HCO₃⁻ reabsorption. The relevant amount of active H⁺ secretion needed for appropriate urine acidification locates to the collecting duct (CD) specific α-intercalated cells (α-IC). These cells express H⁺- and H⁺/K⁺-pumps that conduct energy-consuming H⁺ secretion. In rats, the processes of H⁺ secretion result in a reduction in pH from ~7.4 in the filtrate to PH ~6.5 in the fluid at the transition between the TAL and the distal tubular system. In parallel, a reduction in HCO₃⁻ concentration from ~24 mM in the filtrate to near zero at the entry to the distal tubular system occurs. H⁺ secretion in the CD can further reduce pH so the final urine has pH as low as 4.2 in mice and 4.5 in humans. Whether H⁺ secretion proximal of the collecting duct is adaptable to any metabolic demand is unanswered. In contrast, it is established that the α-ICs are highly responding to changing metabolic demands.

1.3. Ammoniagenesis and Tubular NH₃/NH₄⁺ Transport.

Ammoniagenesis takes place in the PT cells. The amino acid glutamine is metabolized by enzymatic activity first to glutamate and later α-ketoglutarate. Both steps generate one NH₃/NH₄⁺. NH₃/NH₄⁺ is subsequently secreted to the tubule lumen resulting in augmented proton buffer capacity of the tubular fluid. Multiple enzymatic steps are physiologically regulated during metabolic acidosis that augments ammoniagenesis. This includes cellular uptake mechanisms for glutamine, glutamine and glutamate metabolic enzymes (glutaminase and glutamate dehydrogenase) and the apical NH₄⁺ transport protein (NHE3).

In contrast to other metabolites secreted from the PT cells, NH₄⁺ does not only “go with the flow” to stay in the tubular fluid until elimination in the final urine. A substantial amount of tubular NH₄⁺ is reabsorbed in the TAL, where it accumulates in the peritubular space to later be re-secreted into the medullary CD. This process is known as the medullary shortcut and requires defined cellular activity in the TAL and the CD.

This implies that the reduced ability to excrete NH₄⁺ in CKD could originate from impeded production in the PT and/or from reduced medullary shortcut ability. The signaling and transduction events that finally regulate NH₄⁺ excretion are unknown.

### 1.4. H⁺ Secretion in the Collecting Duct.

H⁺ secretion in the CD is a task of the α-IC. H⁺ secretion is mediated by primary active transport via either the vacuolar H⁺-ATPase (V-ATPase) or the gastric and colonic isoform of the H⁺/K⁺-ATPase. H⁺ secretion activity also depends on the basolateral extrusion of HCO₃⁻. The H⁺ secreting activity of the α-IC is adaptable to systemic demands and this regulation likely involves regulatory elements on both apical and basolateral ion transporters. Hormonal regulators include angiotensin and aldosterone. However, common for these hormones is that their main homeostatic functions relate to blood pressure, and water and salt balance. They are not considered primary acid/base balance hormones. Thus, the signaling and transduction events stimulating H⁺ secretion in the α-IC during acidosis remain unknown.

1.4. HCO₃⁻ Secretion in the Collecting Duct.

During conditions of alkalosis (too much base in the organism) the kidneys are able to secrete bicarbonate into the urine. This is a task of the β-IC of the collecting duct. These cells express pendrin, a HCO₃⁻/Cl⁻ exchanger in the luminal membrane that propels HCO₃; from the cells to the urine compartment.

### 1.5 Summary of Background of the Invention

The basic conceptual idea is that the CKD kidney has a reduced capacity for NH₄⁺ excretion while the ability to acidify or add base to the urine is intact. Under normal conditions, NH₄⁺ concentration in the urine increases sharply when the amount of free protons increases (decreasing pH) in the urine (see FIG. 1). Both the increased NH₄⁺ excretion and the acidification (and possible addition of base) to the urine are tightly regulated. The regulatory mechanism (hormonal, nervous, etc.) is not known, but the functions are in the healthy kidney completely balanced according to the systemic demand for acid/base excretion. Thus, low NH₄⁺ concentrations are naturally occurring when the need for acid excretion is limited. However, this also means that one cannot interpret a low NH₄⁺ concentration in urine from a CKD kidney as being abnormal or problematic. Any low NH₄⁺ concentration must be interpreted against the need for acid excretion.

The demand for acid excretion can be assessed on the basis of the normally functioning acid/base excretion processes in the CKD kidney. These are:
urinary acidification (pH),
the degree of protonation of titratable bases (expressed either as titratable acid (TA) or titratable base (base buffers (BB) that are not protonated in the final urine) or
when demand for acid excretion is very low the appearance of the base bicarbonate in the urine.

In summary, chronic kidney disease (CKD) comprises a heterogeneous group of conditions leading to gradual loss of kidney function. With time, CKD progresses unescapably to end-stage renal disease, requiring dialysis or a kidney transplant. A clinical hallmark of CKD is acid retention (metabolic acidosis) caused by a reduced capacity to excrete acid via the kidneys. This metabolic acidosis poses an additional strain on the renal tissue that becomes a driver of further organ damage and eventually renal failure. Full-blown metabolic acidosis can be seen by disturbed acid-base parameters in a blood sample.

Many CKD patients develop acid-mediated organ injury despite normal blood acid/base parameters. Identifying these individual patients with an unapparent acid overload, termed subclinical acidosis, preclinical acidosis or eubicarbonatemic acidosis, is currently not a part of clinical practice. However, these patients would benefit from the therapy offered to those with fully developed CKD acidosis. The rationale is to protect the remaining renal function of CKD patients by reducing the acid excretion workload to delay further renal function loss.

As mentioned, in clinical practice, no procedure allows sufficient assessment or quantification of acid retention in chronic kidney disease (CKD) patients. The final consequences of persistent acid retention in late stages of kidney failure can be measured by blood gas analysis as metabolic acidosis.

Herein is presented a diagnostic urinary biomarker concept that enables the detection of renal acid retention in chronic kidney disease (CKD) prior to the development of systemic overt acidosis. A fully developed and validated urine acid-base biomarker concept is presented that is able to surprisingly identify patients with acid retention before they develop systemic acidosis.

Hence, an improved method of identifying individual CKD patients with SA would be advantageous, and in particular a more efficient and/or reliable method and/or a more efficient and/or reliable non-invasive method of identifying individual CKD patients with SA would be advantageous.

Accordingly, an accurate and non-invasive method for identifying CKD individuals having SA or being at risk of developing metabolic acidosis/chronic metabolic acidosis would be advantageous.

### SUMMARY OF THE INVENTION

As mentioned, in clinical practice, no procedure allows sufficient assessment or quantification of acid retention in chronic kidney disease (CKD) patients. Thus, there is an unmet need for such sufficient assessment or quantification.

Herein is presented a diagnostic urinary biomarker concept that enables the detection of renal acid retention in chronic kidney disease (CKD) prior to the development of systemic acidosis (SA). A fully developed and validated urine acid-base biomarker concept is presented that is able to surprisingly identify patients with acid retention before they develop systemic acidosis.

In particular, it is an object of the present invention to provide a method based on urine measurements that can be used to determine if a CKD patient suffers from systemic acidosis or not and if so, to use this information to e.g. begin early treatment or either follow the outcome of a treatment/intervention in said CKD patient or to follow the progression of the disease.

Further, it is an object of the present invention to provide an improved method of identifying individual CKD patients with SA or non-SA as an easy, non-invasive and reliable methods.

Thus, a first aspect of the invention relates to a method for determining the risk of a subject suffering from chronic kidney disease (CKD) having subclinical acidosis (SA), the method comprising
a) measuring or sensing in a urine sample from a subject, the level of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻;
b) determining a score based on a relationship between the levels of the at least two biomarkers by comparing to a reference level; and
c) classifying the score as a positive or negative score;
   wherein said subject is
at risk of having subclinical acidosis, if said score is negative; and
not at risk of having subclinical acidosis, if said score is positive.

Thus, a second aspect of the invention relates to a method for monitoring the development of subclinical acidosis (SA) in a subject suffering from chronic kidney disease (CKD), the method comprising
determining a first score based on a relationship between the levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻, said biomarkers being measured or sensed in a first urine sample from the subject;
determining a second score based on a relationship between the levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻, said biomarkers being measured or sensed in a second urine sample from the subject, wherein the second sample has been obtained at a later time point than the first sample;
comparing corresponding scores in the first and second sample;
wherein
   a negative score in the second sample compared to a positive score in the first sample is indicative of development of subclinical acidosis;
   a positive score in the second sample compared to positive score in the first sample is indicative of no development of subclinical acidosis;
   a negative score in the second sample compared to a negative score in the first sample is indicative of maintained subclinical acidosis;
   a positive score in the second sample compared to negative score in the first sample is indicative of improved/lessening of subclinical acidosis.

Thus, a third aspect of the invention relates to a method for determining the effect of a treatment protocol against subclinical acidosis (SA) for a subject suffering from chronic kidney disease (CKD), the method comprising
determining a first score based on a relationship between the levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻, said biomarkers being measured or sensed in a first urine sample from the subject;
determining a second score based on a relationship between the levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻, said biomarkers being measured or sensed in a second urine sample from the subject, wherein the second sample has been obtained at a later time point than the first sample;
   wherein the treatment protocol has been initiated or completed before the sampling of the first sample or initiated, continued or completed between the sampling of the first and second sample,
comparing scores of the first sample and the second sample; wherein
   a negative score in the second sample compared to a positive score in the first sample is indicative of the treatment protocol not being effective against subclinical acidosis;
   a negative score in the second sample compared to a negative score in the first sample is indicative of the treatment protocol not being effective against subclinical acidosis;
   a positive score in the second sample compared to negative score in the first sample is indicative of the treatment protocol being effective against subclinical acidosis.

A fourth aspect of the present invention is to provide use of urine sample levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻ as biomarkers for determining the risk of a subject of having subclinical acidosis.

A fifth aspect of the invention relates to use of urine sample levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻ as biomarkers for determining the risk of a subject of having subclinical acidosis to predict serious clinical event-free survival of said subject.

### BRIEF DESCRIPTION OF THE FIGURES

FIG. 1—physiological response to increased acid demand for acid excretion in CKD patients and healthy controls

FIG. 1 shows the relationship between urinary NH₄⁺ concentration and urinary pH in 24 h urine samples from CKD patients and healthy control participants. Number of observations: 214 CKD and 82 control urine samples.

[NH₄⁺]ᵤ and pH Relationship

FIG. 2—urinary NH₄⁺ concentration versus urinary pH quantify a systemic acid overload

FIG. 2: A) shows the relationship between urinary NH₄⁺ concentration and urinary pH with a linear cut-off line between the score of SA or non-SA. B) shows development of GFR among CKD patients scored SA or non-SA.

FIG. 3: Dietary intervention on the relationship between urinary NH₄⁺ concentration and urinary pH

FIG. 3: A) show the relationship between urinary NH₄⁺ concentration and urinary pH with a linear cut-off line between the score of SA or non-SA. Black dots are baseline and grey dots after 1-week acid-reducing dietary intervention where two measurements from each patient are linked with thin lines. B) Proportions of CKD patients scored non-SA during a one-week acid-reducing dietary intervention. C) Total plasma CO₂ as a measure of systemic acid/base status in the blood. D) Proportions of fully acidotic patients scored non-acidotic during a one-week acid-reducing dietary intervention.

FIG. 4—Event-free survival probability of SA and non-SA scored CKD patients (Urine NH₄⁺ vs. urinary pH)

FIG. 4 show a Kaplan-Meyer blot displaying the probability of serious clinical event-free survival (further 50% reduction in GFR, entering dialysis or kidney transplantation) of the SA and non-SA scored CKD patients during 7 years follow-up. Patients scored as SA or non-SA based on urinary [NH₄⁺] pH relationship and the cut-off displayed in FIG. 2A.

FIG. 5: Relationship between urinary NH₄⁺ concentration and urinary pH

FIG. 5: A) shows the relationship between urinary NH₄⁺ concentration and urinary pH with the non-linear cut-off equation between the score of SA or non-SA. B) Development of GFR among CKD patients scored SA or non-SA at follow-up 1.5 years post scoring. C) Development of eGFR (estimated GFR) among CKD patients scored SA or non-SA during 7 years follow-up.

FIG. 6: Relationship between urinary NH₄⁺ concentration and urinary pH

FIG. 6: A) Relationship between urinary NH₄⁺ concentration and urinary pH with the non-linear cut-off equation between the score of SA or non-SA. Black dots are baseline and grey dots after 1-week acid-reducing dietary intervention. B) Proportions of CKD patients scored non-SA during a one-week acid-reducing dietary intervention.

FIG. 7: Event-free survival probability of SA and non-SA scored CKD patients (Urine NH₄⁺ vs. urinary pH)

FIG. 7: Kaplan-Meyer blot displaying the probability of serious clinical event-free survival (further 50% reduction in GFR, entering dialysis or kidney transplantation) of the SA and non-SA scored CKD patients during 7 years follow-up. Patients scored as SA or non-SA based on urinary [NH₄⁺] pH relationship and the non-liniar cut-off equation displayed in FIG. 5A.

[NH₄⁺]ᵤ and [TA] Relationship

FIG. 8: Relationship between urinary NH₄⁺ concentration and urinary TA

FIG. 8: A) Relationship between urinary NH₄⁺ concentration and urinary TA with the non-linear cut-off equation between the score of SA or non-SA. B) Development of estimated GFR among CKD patients scored SA or non-SA, wherein the development was followed for 6 years.

FIG. 9—Event-free survival probability of SA and non-SA scored CKD patients (Urine NH₄⁺ vs. urinary TA)

FIG. 9: Kaplan-Meyer blot displaying the probability of serious clinical event-free survival (further 50% reduction in GFR, entering dialysis or kidney transplantation) of the SA and non-SA scored CKD patients during 7 years follow-up. Patients scored as SA or non-SA based on urinary [NH₄⁺] vs TA relationship and the cut-off equation displayed in FIG. 8A.

NH₄⁺ and Total Base (TB) Excretion Relationship

FIG. 10—Relationship between urinary NH₄⁺ excretion and urinary total base excretion rate

FIG. 10: A) Relationship between urinary NH₄⁺ excretion and urinary total base excretion rate (TB=([BB]+ [HCO₃⁻])*urine volume) with the linear cut-off line between the score of SA or non-SA. B) Development of GFR among CKD patients scored SA or non-SA at follow-up 1.5 years post scoring.

FIG. 11—Relationship between urinary NH₄⁺ excretion and urinary TB excretion

FIG. 11: A) Relationship between urinary NH₄⁺ excretion and urinary TB excretion with the linear cut-off line between the score of SA or non-SA. Black dots are baseline and grey dots after 1-week of acid-reducing dietary intervention. B) Proportions of CKD patients scored non-SA during a one-week acid-reducing dietary intervention.

FIG. 12—Event-free survival probability of SA and non-SA scored CKD patients (urinary NH₄⁺ excretion vs urinary total base excretion)

FIG. 12: Kaplan-Meyer blot displaying the probability of serious clinical event-free survival (further 50% reduction in GFR, entering dialysis or kidney transplantation) of the SA and non-SA scored CKD patients during 7 years follow-up. Patients scored as SA or non-SA based on the cut-off equation displayed in FIG. 11A.

The Relative (Normalized to Creatinine) NH₄⁺ and Base Buffer/Bicarbonate Excretion Relationship

FIG. 13—Relationship between urinary NH₄⁺ creatinine ratio and BB creatinine ratio. Relationship between urinary NH₄⁺ creatinine ratio and HCO₃-creatinine ratio

FIG. 13: A) Relationship between urinary NH₄⁺ creatinine ratio and BB creatinine ratio with the suggested cut-off line between the score of SA or non-SA.

B) Relationship between urinary NH₄⁺ creatinine ratio and HCO₃-creatinine ratio with the suggested cut-off line between the score of SA or non-SA.

FIG. 14—Event-free survival probability of SA and non-SA scored CKD patients (urinary NH₄⁺ creatinine ratio vs BB creatinine ratio)

FIG. 14: Kaplan-Meyer blot displaying the probability of serious clinical event-free survival (further 50% reduction in GFR, entering dialysis or kidney transplantation) of the SA and non-SA scored CKD patients during 7 years follow-up. Patients scored as SA or non-SA based on the linear cut-off equation displayed in FIG. 13A.

FIG. 15—Event-free survival probability of SA and non-SA scored CKD patients (urinary NH₄⁺ creatinine ratio vs HCO₃-creatinine ratio)

FIG. 15: Kaplan-Meyer blot displaying the probability of serious clinical event-free survival (further 50% reduction in GFR, entering dialysis or kidney transplantation) of the SA and non-SA scored CKD patients during 7 years follow-up. Patients scored as SA or non-SA based on the cut-off equation displayed in FIG. 13B.

FIG. 16—Characterization of the AB_score

A) AB_score of controls and CKD patients. AB_score cut-off between SA and non SA is the lower 2.5th percentile of control participants. B) urine pH and [NH₄⁺] in urine samples from controls (n=25) and CKD patients (n=82). The black lines indicate AB_score cut-off. C) Association between measured GFR at inclusion and AB_score. D) Association between total plasma CO₂ at inclusion and AB_score. E) 9 consecutive AB_score assessments in 28 CKD patients. F) Intra-individual coefficient of variation of AB_score in 9 consecutive AB score assessments (n=28).

FIG. 17—Characterization of the AB_score

Calculated AB_scores in control participants (RENVAS, n=25), development (RENVAS, n=82), validation cohort (PUMA, n=73), and variation cohort (NNRD, n=58). Please note that there was no systematic difference in AB_score between CKD cohorts

FIG. 18—Characterization of the AB_score

A) Calculated AB_scores in 24 h urine and spot urine collections in the validation cohort (PUMA). B) Assessment of AB_score in 24 h plotted against assessment of AB_score in spot urine collection from each participant. Note no systematic bias between the 24 h and spot urine assessments was identified. C) Bland-Altmann plot for spot vs. 24 h urine AB_score assessment. D) Intra-individual coefficient of variance of AB_score in between 24 h urine and spot urine collections.

FIG. 19—CKD progression in developmental cohort

AB_score associates with CKD progression in the developmental cohort (RENVAS) A) Percentage change in measured GFR from inclusion and 18 months after inclusion in CKD patients scored non-SA or SA. Note the significant reduction in mGFR in the SA score CKD patients. Error-bars represent 95% CI intervals. B) Kaplan Meier plot displaying event-free survival in the lowest, middle and highest AB_score tertile. C) Kaplan-Meier plot displaying event-free survival among CKD patients scored non-SA and SA. Please note the low frequency of events among non-SA CKD patients.

FIG. 20—CKD progression in the validation cohort

Kaplan Meier plots display event-free survival in A) the lowest, middle, and highest AB_score tertile and B) non-SA and SA CKD patients in the validation cohort (PUMA) Please note the low frequency of clinical events among non-SA CKD patients.

FIG. 21—Analysis of pooling two cohorts

A cubic spline graph (3 knots) for the association between AB_score and the hazard ratio for CKD progression in a pooled analysis of RENVAS and PUMA. The cut-off based on the 2.5th percentile of control participants was used as reference point

The present invention will now be described in more detail in the following.

### DETAILED DESCRIPTION OF THE INVENTION

a Method for Determining the Risk of a Subject Suffering from Chronic Kidney Disease (CKD) Having Subclinical Acidosis
A scoring system that assesses the systemic need/demand to excrete acid in relation to the remaining ability of the diseased kidney to excrete acid equivalents

Several studies have shown that a low renal NH₄⁺ excretion is associated with faster progression of CKD and a higher risk of poor renal outcomes, i.e. end-stage renal disease. Without being bound by theory, this could make physiological sense, as a low urine NH₄⁺ excretion could cause acid retention, which also associates with worse renal outcomes. Conversely, while a low NH₄⁺ excretion could reflect a low capacity of the kidneys to generate and excrete NH₄⁺, it could also reflect a lower need for NH₄⁺ excretion. Also, the treatment options for acid retention are acid-reducing dietary regimes, base supplementation, pharmacological treatment or gastrointestinal proton chelators. These treatments will evidently not increase NH₄⁺ excretion (likely decrease NH₄⁺ excretion) but instead decrease the need to excrete NH₄⁺ so that the workload of the kidney is kept within its diseased working range. Hence, NH₄⁺ excretion alone cannot be a good parameter to assess treatment response. It could be argued that systemic acid-base status would be a good choice to monitor treatment efficacy, but there are no clear guidelines as to what the tCO₂ (or stdHCO₃⁻) level should be. With this in mind, the present inventors have developed scoring systems that assess the systemic need/demand to excrete acid in relation to the remaining ability of the diseased kidney to excrete NH₄⁺. In situations where the demand for acid excretion can not be matched by sufficient NH₄⁺ excretion, patients are scored as subclinical acidotic (SA). Opposite, non-SA-scored patients match their need for acid excretion with sufficient NH₄⁺ excretion.

### Method Summary:

In conditions where the human kidneys need to excrete acid, urine pH (or any urine acid/base biomarker that correlates directly with the pH of the urine e.g., total urinary base, urinary base buffers, and urinary HCO₃⁻) will decrease. All pH-dependent biomarkers reach very low levels (for TB and BB even negative) as the demand for acid excretion increases. Concurrently, proximal tubule ammoniagenesis and NH₄⁺ excretion increase dramatically. In CKD patients, the ability to generate and excrete NH₄⁺ is compromised. Thus, the present inventors used urine pH, TB or BB as indicators of the need to excrete acids and NH₄⁺ as the capacity to do so.

Thus, the present inventors have developed urine acid-base scoring concepts or “AB_score” that assess the capacity to excrete NH₄⁺ adjusted for the need to eliminate acid equivalents. In the following, six relevant scoring concepts are presented along with a variant of scoring concept 2 called “AB_score”:

### Scoring Concept 1:

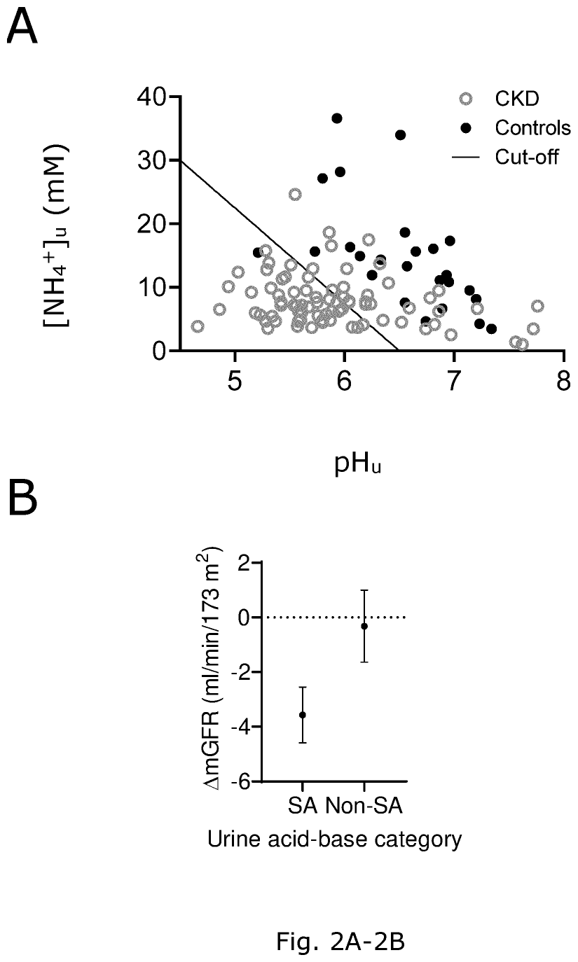

Based on the [NH₄⁺]ᵤ and pH relationship and a linear cut-off between SA and non-SA. (FIG. 2-4)
non-SA when NH₄⁺≥−15*pHᵤ+97.5
or non-SA when pHᵤ≥6.5

### Scoring Concept 2 or “AB_Score”:

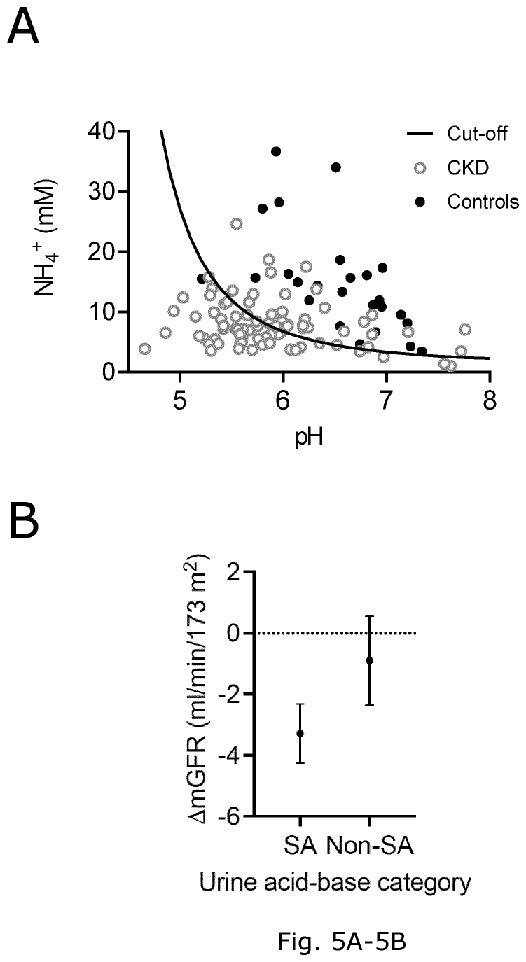

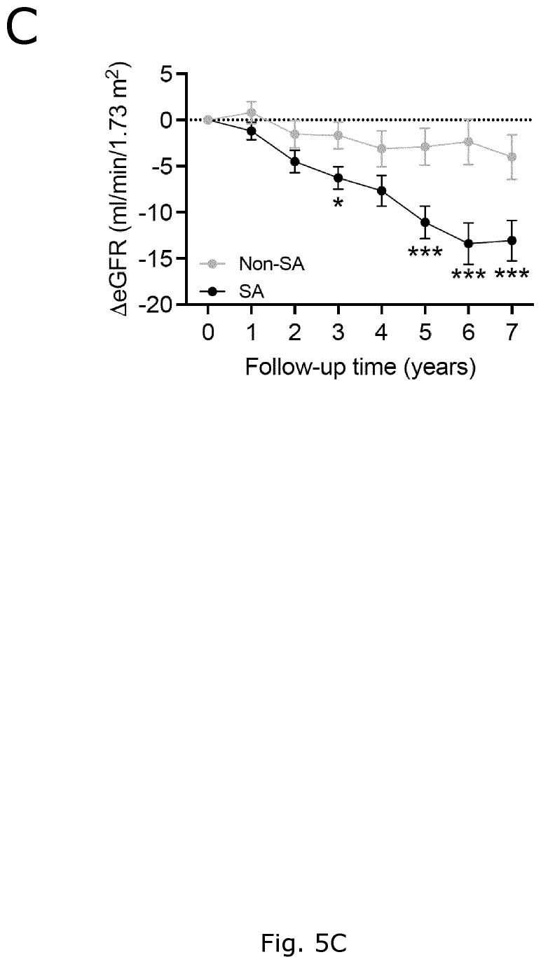

Based on the [NH₄⁺]ᵤ and pH relationship and a non-linear cut-off equation between SA and non-SA. (FIG. 5-7)
non-SA when log ([NH₄⁺]ᵤ)*(pHᵤ³)/10>17.93
or non-SA when ((log ([NH₄⁺]ᵤ)*(pHᵤ³)/10))−17.93>0
   log ([NH₄⁺]) was used as the relationship between pH and NH₄⁺ is non-linear. pH was raised to the power of 3 to allow a higher urine pH to affect the score more than a low urine pH. Thus, at low urine pH, NH₄⁺ is weighed higher and at high pH, pH is weighed higher. Hence, a positive (high) score can be accomplished by having a higher need to excrete acid equivalents with a concurrent high urine NH₄⁺ or a lower need to excrete acid equivalents. The cut-off (17.93) was based on the lower limit of the 95% prediction interval in control participants.

A preferred way to present scoring concept 2 is shown by the following formula:

$$AB\_score = \frac{log [ {NH}_{4}^{+} ] \cdot {pH}^{3}}{10}$$

### The Name “AB Score” is Short for a Urine Acid/Base Score:

The equation provides the strongest association between the urine AB_score and both urinary [NH₄⁺] and urinary pH. In an embodiment, a cut-off of 19 provided a good AB score.

### Scoring Concept 3:

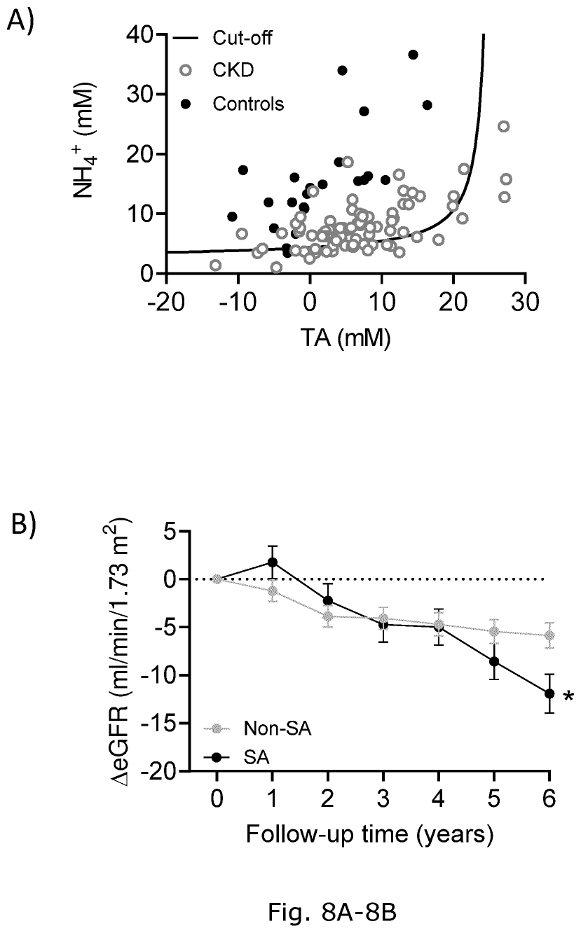

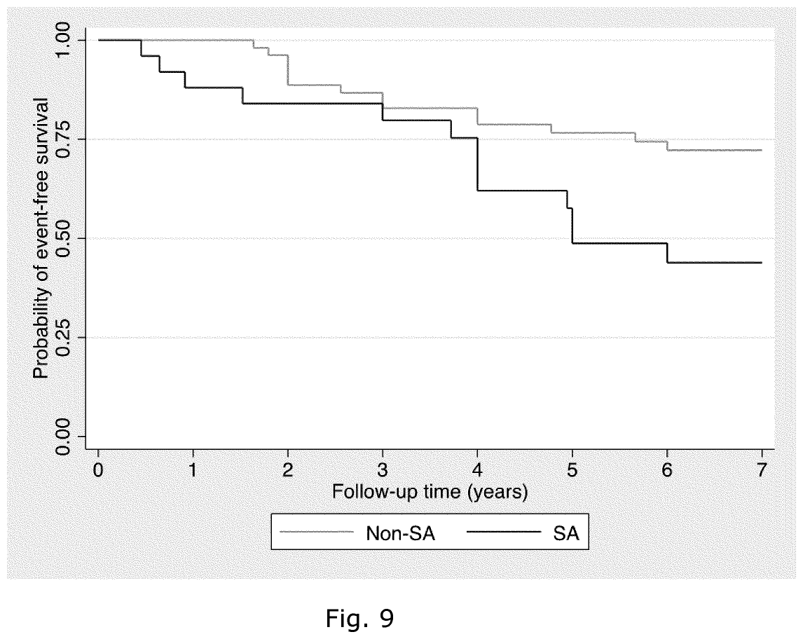

Based on the [NH₄⁺]ᵤ and [TA] relationship and a non-linear cut-off equation between SA and non-SA. (FIGS. 8 and 9)
non-SA when log [NH₄⁺]ᵤ*log (([TA]ᵤ*−1)+28)<0.926
or non-SA when (log [NH₄⁺]ᵤ*log (([TA]ᵤ*−1)+28)−0.926<0

28 is added to avoid negative TA values, to allow log transformation. It will of course depend on what TA is (if TA is lower than −28, more than 28 needs to be added)

### Scoring Concept 4:

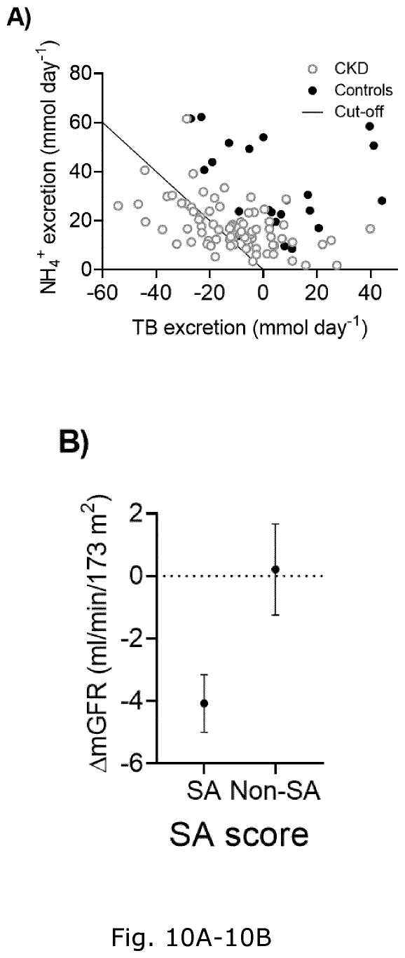

Based on the relationship between NH₄⁺ and total base excretion and a linear cut-off between SA and non-SA (FIG. 10-12).
non-SA when NH₄⁺ excretion≥−1*TB excretion+0
or non-SA when TB excretion is ≥0

### Scoring Concept 5:

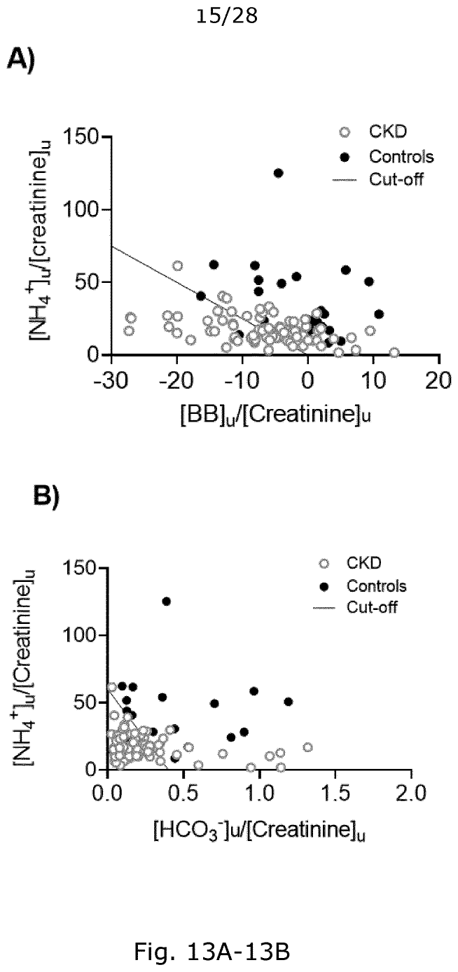

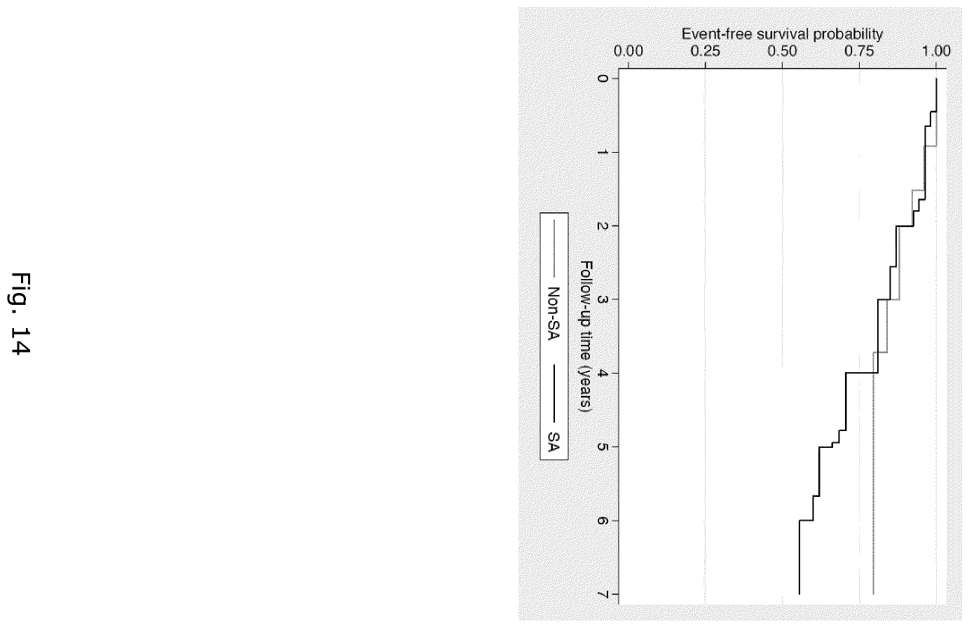

Based on the relationship between the relative (normalized to creatinine) NH₄⁺ excretion and relative base buffer excretion and a linear cut-off between SA and non-SA (FIGS. 13a and 14):
non-SA when [NH₄⁺]ᵤ/[creatinine]ᵤ>−2.5*[BB]ᵤ/[Creatinine]ᵤ+0
or non-SA when [BB]ᵤ/[Creatinine]ᵤ is ≥0

### Scoring Concept 6:

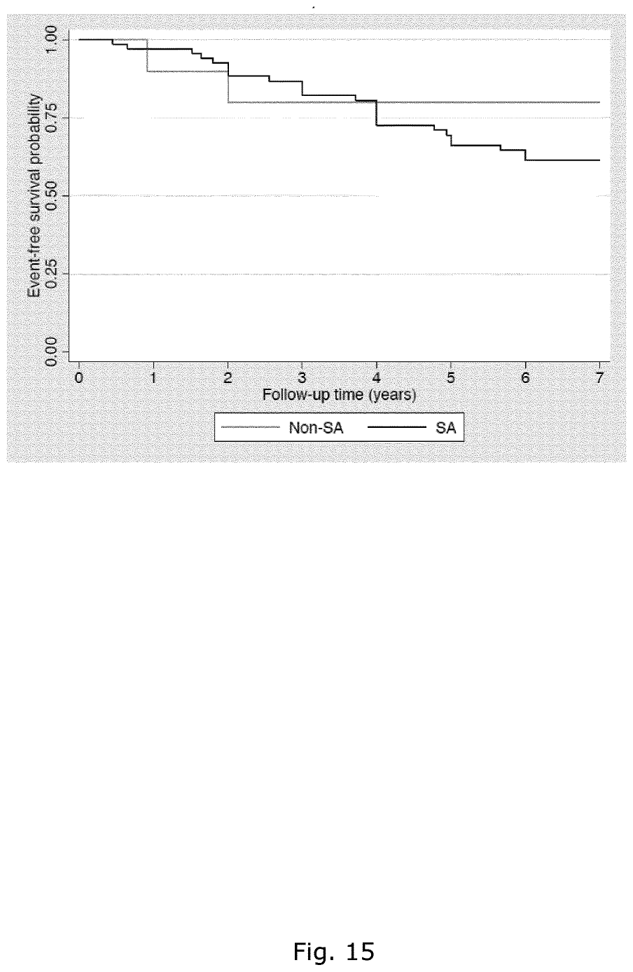

Based on the relationship between the relative (normalized to creatinine) NH₄⁺ excretion and relative HCO₃-excretion and a linear cut-off between SA and non-SA (FIGS. 13b and 15):
non-SA when [NH₄⁺]ᵤ/[creatinine]ᵤ>−150*[HCO₃]ᵤ/[Creatinine]ᵤ+0
or non-SA when [HCO₃]ᵤ/[Creatinine]ᵤ is ≥0

The above-mentioned scoring concepts are examples of ways to analyse the relationship between at least two relevant biomarkers for CKD patients but are not a complete list as other mathematical formulas, software/AI may be used to analyse this relationship.

The core of the invention is indeed the general concept of having developed urine acid-base scoring concepts that assess the capacity to excrete NH₄⁺ adjusted for the need to eliminate acid equivalents.

This general concept surprisingly allows the division of CKD patients into two groups having either subclinical acidosis (SA) or non-SA i.e. not having subclinical acidosis.

Accordingly, said general concept allows the detection of renal acid retention in chronic kidney disease (CKD) patients prior to the development of systemic acidosis/metabolic acidosis. Thus, a fully developed and validated urine acid-base biomarker concept is presented that is able to surprisingly identify patients with acid retention before they develop metabolic acidosis. The method is non-invasive, reliable og flexible.

### Definitions

Prior to discussing the present invention in further detail, the following terms and conventions will first be defined:

### Chronic Kidney Disease (CKD)

“CKD” as used herein refers to chronic kidney disease. The disease is divided into various well-defined stages, i.e. grades, such as e.g. grade G1. The CKD grades are described in the table below.

TABLE 1
CKD grades

| CKD | Description of | GFR |
|---|---|---|
| grades | kidney function | (ml/min/1.73 m²)* |
| G1 | Normal or high kidney function | ≥90 |
| G2 | Mildly decreased | 60-89 |
| G3a | Mildly to moderately decreased | 45-59 |
| G3b | Moderately to severely decreased | 30-44 |
| G4 | Severely decreased | 15-29 |
| G5 | Kidney failure | <15 |

*GFR = glomerular Filtration Rate

### “CKD Acidosis” or “CKD Metabolic Acidosis or “Metabolic Acidosis”

“CKD acidosis” is short for “CKD metabolic acidosis” or “metabolic acidosis” and all terms are used interchangeably and when used herein refers to CKD patients having a fully developed acidosis. In other words, said CKD patients are “acidotic”. Thus, “metabolic acidosis” refers to a condition in which a CKD patient has a plasma HCO₃-concentration or plasma total CO₂ (tCO₂) of <22 mM i.e. a level in which the body has an acid content that is too high to support good health. Once this level is achieved, treatment of said CKD patients is advised i.e. the treatment options for acid retention are acid-reducing dietary regimes, base supplementation (e.g. peroral bicarbonate supplementation), or gastrointestinal proton chelators to protect the remaining renal function of CKD patients by reducing the acid excretion workload to delay further renal function loss. The threshold plasma HCO₃⁻; or tCO₂ levels are determined by KDIGO (a global organization developing and implementing evidence-based clinical practice guidelines in kidney disease).

### No Apparent Metabolic Acidosis

“No apparent metabolic acidosis” as used herein refers to a CKD patient that meets the standard HCO₃ plasma concentration≥22 mmol/l or total CO₂ under 22 mmol/l). CKD patients that have no apparent metabolic acidosis are used as the inclusion criteria for selection of CKD patients that comprise a normal systemic acid/base status, i.e. that have no apparent metabolic acidosis.

### Normal Systemic Acid/Base Status

“Normal systemic acid/base status” as used herein refers to CKD patients that have no apparent metabolic acidosis.

### “SA” or “Subclinical Acidosis”

“SA” or “subclinical acidosis” as used herein refers to CKD patients with an unobvious acid overload/retention, which is termed subclinical acidosis (SA), preclinical acidosis or eubicarbonatemic acidosis. This condition is currently not approached in clinical practice but many CKD patients develop acid-mediated organ injury despite normal blood acid/base parameters.

Thus, in situations where the demand for acid excretion can not be matched by sufficient NH₄⁺ excretion, patients are scored as subclinical acidotic (SA).

### “Non-SA”

“Non-SA” as used herein refers to CKD patients with no acid overload, which is termed non-subclinical acidosis (non-SA),

Thus, in situations where the demand for acid excretion can be matched by sufficient NH₄⁺ excretion, patients are scored as non-subclinical acidotic (non-SA). Thus, non-SA-scored patients match their need for acid excretion with sufficient NH₄⁺ excretion.

### “Subclinical Acidosis Score”

“Subclinical acidosis score” or shortened to simply “score” as used herein refers to a subclinical acidosis score that is provided by the ratios of measured or sensed acid/base parameters in urine samples or the relationship between measured or sensed acid/base parameters in urine samples. By “measuring” or “sensing” is also meant “determining” values in the urine. In some embodiments, calculating values in the urine may also be an option. The measured or sensed parameters may be at least one of the biomarkers urinary NH₄⁺, TA, pH, TB, BB and HCO₃⁻. The calculations of these parameters/biomarkers are shown in example 1. Further, the subclinical acidosis score/scoring concept may also be provided by use of artificial intelligence (“AI”) as defined briefly below.

### “Software/“AI”

Another embodiment of the present invention relates to processor system programmed to operate according to a machine learning (ML) algorithm for estimating the score based on a relationship between the levels of biomarkers of the invention, the machine learning (ML) algorithm being trained, and/or being trainable, on data obtained by a method according to the first aspect of the invention or the second aspect of the invention or the third aspect of the invention. Preferably, said biomarkers of the invention are two biomarkers, even more preferably said two biomarkers are NH₄⁺ and pH or they may be at least one of the urinary biomarkers NH₄⁺ vs. titratable acid (TA), NH₄⁺ vs. total base (TB), NH₄⁺ vs. base buffers (BB), and/or NH₄⁺ vs. HCO₃⁻.

In yet another embodiment, the invention relates to use of a machine learning (ML) algorithm trained on data obtained by a method according to the first or second or third aspect of the invention to predict the score based on a relationship between the levels of biomarkers of the invention. Preferably, said biomarkers of the invention are two biomarkers, even more preferably said two biomarkers are NH₄⁺ and pH or they may be at least one of the urinary biomarkers NH₄⁺ vs. titratable acid (TA), NH₄⁺ vs. total base (TB), NH₄⁺ vs. base buffers (BB), and/or NH₄⁺ vs. HCO₃⁻.

An embodiment of the present invention relates to a system suitable for executing an algorithm (such as machine learning (ML) algorithm) for estimating the score based on a relationship between the levels of biomarkers of the invention, the (machine learning) system being trained, and/or being trainable, on data provided according to the first, second or third aspect of the invention.

Advantageously, the invention may also relate to a method for training a machine learning (ML) system for estimating the score based on a relationship between the levels of biomarkers of the invention, such as according to the the first, second or third aspect of the invention.

Thus, yet an embodiment of the invention relates to a method comprises the steps of:
receiving training data comprising a first set of information (1SI), such as a first database, and a second set of information (2SI), such as a second database,
training the system for estimating score variants using said training data, and
validating the system using correlated specific score variants to the frequency information, such as frequency of the variant.

Preferably the system and/or algorithm and/or method is implemented on a computer, thus being computer-implemented.

Below is given a list of some no-limiting types of algorithms that are particularly suited for machine learning (ML) system and/or training of a (ML) system using urine data:
1. Deep Learning Algorithms: Deep learning is a subset of machine learning where artificial neural networks, algorithms inspired by the human brain, learn from large amounts of data. Deep learning algorithms are capable of learning to represent the world as a nested hierarchy of concepts, with each concept defined in relation to simpler concepts, and more abstract representations computed in terms of less abstract ones. The skilled reader is referred to for example University of Illinois at Urbana-Champaign; “AI predicts enzyme function better than leading tools.” ScienceDaily. ScienceDaily, 30 Mar. 2023. <www.sciencedaily.com/releases/2023/03/230330172121.htm>.
2. Contrastive Learning: This is a type of unsupervised learning approach that trains models to learn similar features from similar data points and different features from different data points. An AI tool named ‘CLEAN’ was recently reported to use this algorithm to predict enzyme function, cf. Gupta, R., Srivastava, D., Sahu, M. et al. Artificial intelligence to deep learning: machine intelligence approach for drug discovery. Mol Divers 25, 1315-1360 (2021) for more details.
3. Artificial Neural Networks (ANNs): ANNs are computing systems vaguely inspired by the biological neural networks that constitute animal brains. An ANN is based on a collection of connected units or nodes called artificial neurons, which loosely model the neurons in a biological brain.
4. Support Vector Machines (SVMs): SVMs are supervised learning models with associated learning algorithms that analyze data for classification and regression analysis.
5. Generative Adversarial Networks (GANs): GANs are a class of artificial intelligence algorithms used in unsupervised machine learning, implemented by a system of two neural networks contesting with each other in a zero-sum game framework.

These algorithms can be used individually or in combination, depending on the specific requirements of the type of enzyme data. The invention according to this aspect can be implemented by means of hardware, software, firmware or any combination of these. The invention or some of the features thereof can also be implemented as software running on one or more data processors and/or digital signal processors.

The individual elements of an embodiment of the invention may be physically, functionally and logically implemented in any suitable way such as in a single unit, in a plurality of units or as part of separate functional units. The invention may be implemented in a single unit, or be both physically and functionally distributed between different units and processors.

### “a Negative Score” vs. “a Positive Score”

“A negative score” as used herein means that a CKD patient is regarded as having SA, whereas “a positive score” means that a CKD patient is regarded as not having SA i.e. being expressed as non-SA.

### Cut-Off Line

“Cut-off line” as used herein refers to a line that separates SA and non-SA CKD patients. Such a cut-off line can be determined or calculated in a variety of ways, depending on which parameters and calculation methods are used for the line separating the SA and non-SA patients. The cut-off lines may therefore be linear or non-linear. The cut-off calculation methods are called “scoring concepts”. The core of the invention resides in the fact that CKD patients with SA can be divided into a SA and non-SA group thereby allowing clinicians to treat the subclinical acidotic CKD patients before they develop acid-mediated organ injury despite their normal blood acid/base parameters/status.

### Kidney Function Loss (AmGFR)

“Kidney function loss (AmGFR)” as used herein refers to the glomerular filtration rate (GFR) measured at the first clinical visit subtracted by measured GFR at the second clinical visit (18 months later).

“Measured Glomerular Filtration Rate (mGFR)”

“Measured glomerular filtration rate (mGFR)” as used herein refers to the clinical standard way to measure GFR. GFR is measured as the renal radioactive ⁵¹Cr-EDTA clearance. GFR is considered a mostly reliable test for doctors to know how well your kidneys are working.

eGFR

“eGFR” as used herein refers to estimated glomerular filtration rate. eGFR is an estimated number based on the blood level of creatinine and the humans age, sex of the respective patient. eGFR is cost and time-effective assessment of GFR but lack accuracy as compared to mGFR.

### 24 h Urine Collection

“24 h urine collection” as used herein refers to a 24-hour urine collection that is done by collecting the urine of a subject in a special container over a full 24-hour period.

### Spot Urine Sample

“Spot urine sample” as used herein refers to urine samples collected at one-time point during the day. This also means “simple spot urine samples”.

### Titratable Acid (TA)

The acid in human urine is bound in buffers such as sulfates, phosphates and other inorganic anions derived from normal metabolism. “Titratable acid (TA)” or “urinary TA/UTA” as used herein thus refers to protons bound to these anions.

### “Ammonium” and “Bicarbonate”

As used herein, “ammonium” is used interchangeably with “NH₄⁺” and “bicarbonate” is used interchangeably with “HCO₃⁻”.

### Titratable Bases TB

“Titratable bases (TB)” or “urinary TB” as used herein refers to urinary measured TA subtracted from measured HCO₃⁻ i.e. TB=(HCO₃⁻− TA).

### Base Buffers (BB)

“Base buffers (BB)” or “titratable base (base buffers (BB))” as used herein refers to the amount of not protonated anion buffers contained in human urine.

### New Nordic Renal Diet (NNRD)

“New Nordic Renal Diet (NNRD)” as used herein refers to an acid-reducing dietary intervention (New Nordic Renal Diet, NNRD) that is known to reduce the systemic acid load.

### Total Plasma CO₂

“Total plasma CO₂” or “tCO₂” as used herein refers to a standard measure in blood samples. The total plasma CO₂ (tCO₂) is used as a measure to evaluate the systemic acid/base status of a subject. It could be argued that systemic acid-base status would be a good choice to monitor treatment efficacy, but there are no clear guidelines as to what the tCO₂ (or stdHCO₃⁻) level should be.

### Total Base Excretion

“Total base excretion” as used herein is calculated as the urinary total base excretion rate (TB=([BB]+ [HCO₃⁻])*urine volume)

RenVas cohort

“RenVas” as used herein refers to Renal and Systemic Vascular Resistance in Chronic Kidney Disease (CKD) (RenVas) (clinical trials registry number: NCT01380717). Cohort studies refer to a type of longitudinal study—an approach that follows research participants over a period of time (often many years). Specifically, cohort studies recruit and follow participants who share a common characteristic, such as a particular occupation or demographic similarity. Thus, “RenVas cohort” as used herein refers to a long-term study of the disease prognosis of CKD patients. As disclosed herein, the inventors have divided patients into SA and non-SA groups and subsequently have followed their disease progression in a long-term study i.e. the RenVas cohort as demonstrated in Example 1. The measurements were made in a 7 year follow-up in regard to composite endpoint being one or more of the following renal events: Dialysis, kidney transplantation, or a 50% reduction in eGFR.

### Kaplan-Meier Survival Plot

“Kaplan-Meier survival plot” or simply “Kaplan-Meier plot” as used herein is a tool used to estimate the survival function from lifetime data. In medical research, it is often used to measure the fraction of patients living for a certain amount of time after treatment. As used herein, the Kaplan-Meier survival plot is used to display the probability of serious clinical event-free survival (further 50% reduction in GFR, entering dialysis or kidney transplantation) of SA and non-SA scored CKD patients. Hazard ratios are calculated by the well-known “cox proportional hazards model”.

### Reference Level

In the context of the present invention, the term “reference level” relates to a standard in relation to a quantity, which other values or characteristics can be compared to.

In one embodiment of the present invention, it is possible to determine a reference level by investigating the biomarker levels in urine samples from healthy subjects i.e. “controls”. Said biomarker levels may be pH, NH₄⁺, TA, TB, BB or HCO₃⁻. Also, it is possible to determine a reference level by investigating the biomarker levels in urine samples from healthy subjects said biomarker levels being a relationship between pH vs. NH₄⁺, TA vs NH₄⁺, TB vs. NH₄⁺, BB vs NH₄⁺, HCO₃⁻ VS. NH₄⁺, pH vs. HCO₃⁻, and pH vs. TA. By applying different statistical means, such as multivariate analysis, linear or non-linear calculations one or more reference levels can be calculated.

Based on these results, a cut-off may be obtained that shows the relationship between the level(s) detected and patients at risk. The cut-off can thereby be used e.g. to determine whether a CKD patient has SA or non-SA, which, if the patient has SA, for instance, corresponds to an increased risk of having or developing systemic acidosis.

### Risk Assessment

The present inventors have successfully developed a new method to predict the risk of a CKD patient having or developing acid retention. To determine whether a CKD patient has an increased risk of having or developing acid retention, a cut-off (reference level) must be established. This cut-off may be established by the laboratory, the physician or on a case-by-case basis for each patient.

The cut-off level could be established using a number of methods, including the scoring concepts 1-6 as discussed herein.

Statistics enables evaluation of the significance of each level. Commonly used statistical tests applied to a data set include t-test, f-test or even more advanced tests and methods of comparing data. Using such a test or method enables the determination of whether two or more samples are significantly different or not.

The significance may be determined by the standard statistical methodology known by the person skilled in the art.

The chosen reference level may be changed depending on the mammal/subject for which the test is applied.

Preferably, the subject according to the invention is a human subject with CKD, such as a subject considered at risk of having SA and/or acid retention.

The chosen reference level may be changed if desired to give a different specificity or sensitivity as known in the art. Sensitivity and specificity are widely used statistics to describe and quantify how good and reliable a biomarker or a diagnostic test is. Sensitivity evaluates how good a biomarker or a diagnostic test is at detecting disease, while specificity estimates how likely an individual (i.e. control, a patient without disease) can be correctly identified as not at risk.

Thus, a first aspect of the invention relates to a method for determining the risk of a subject suffering from chronic kidney disease (CKD) having subclinical acidosis (SA) or having acid retention, the method comprising
a) measuring or sensing in a urine sample from a subject, the level of at least two biomarkers selected from the group consisting of
NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻;
b) determining a score based on a relationship between the levels of the at least two biomarkers by comparing to a reference level; and
c) classifying the score as a positive or negative score;
   wherein said subject is
at risk of having subclinical acidosis, if said score is negative; and
not at risk of having subclinical acidosis, if said score is positive.

An embodiment of the invention relates to a method for determining the risk of a subject suffering from chronic kidney disease (CKD) having subclinical acidosis (SA) or having acid retention, the method comprising
a) measuring or calculating in a urine sample from a subject, the level of at least two biomarkers selected from the group consisting of NH₄⁺, TA, TB, pH, BB and HCO₃⁻;
b) determining a score based on the levels of the at least two biomarkers by comparing to a reference level; and
c) classifying the score as a positive or negative score;
   wherein said subject is
at risk of having subclinical acidosis, if said score is negative; and
not at risk of having subclinial acidosis, if said score is positive.

A further embodiment of the invention relates to a method for determining the risk of a subject suffering from chronic kidney disease (CKD) having acid retention, the method comprising
a) measuring or calculating in a urine sample from a subject, the level of at least two biomarkers selected from the group consisting of NH₄⁺, TA, TB, pH, BB and HCO₃⁻;
b) determining a score based on the levels of at least two biomarkers by comparing to a reference level; and
c) classifying the score as a positive or negative score;
   wherein said subject is
at risk of having subclinical acidosis or acid retention, if said score is negative; and
not at risk of having subclinial acidosis or acid retention, if said score is positive.

In an embodiment of the invention, a method for determining the risk of a subject suffering from chronic kidney disease (CKD) having subclinical acidosis (SA) or having acid retention is disclosed, the method comprising
a) measuring or calculating in a urine sample from a subject, the level of at least two biomarkers selected from the group consisting of NH₄⁺, TA, TB, pH, BB and HCO₃;
b) determining a score based on the levels of the at least two biomarkers by comparing to a reference level; and
c) classifying the score as a positive or negative score;
   wherein said subject
has subclinical acidosis (SA), if said score is negative; and
does not have subclinial acidosis (non-SA), if said score is positive.

In an embodiment of the invention, measuring or calculating in a urine sample from a subject is a method, wherein the level of at least tree biomarkers are selected from the group consisting of NH₄⁺, TA, TB, BB, PH and HCO₃⁻.

In another embodiment of the invention, measuring or calculating in a urine sample from a subject is a method, wherein the level of at least four biomarkers are selected from the group consisting of NH₄⁺, TA, TB, BB, PH and HCO₃⁻.

In a preferred embodiment of the invention, said urine sample is a 24 h urine collection or a spot urine sample. Example 6 clearly demonstrate the surprising effects of using spot-urine samples from subjects as a method, wherein the level of at least two biomarkers can be measured or calculated.

In an embodiment, the method according to the invention is a method wherein said subject is suffering from chronic kidney disease (CKD).

In an embodiment, said method according to the invention is a method, wherein said subject is at risk of having acid retention.

In an embodiment, said method according to the invention is a method, wherein said subject is suffering from acid retention.

In an embodiment of the invention, said subject is suffering from chronic kidney disease (CKD) selected from grade CKD grade G2, such as G3a, such as G3b, such as G3, such as G4, such as G5, such as G2-G3, such as G2-G4, such as G3-G4, such as G4-G5, such as G3a-G3b, such as G2-G3a, such as G2-G3b, such as G3a-G4, such as G3b-G4, such as G3-G5. Preferably, said subject has CKD G2 or G3, or even more preferably G2-G3.

In an embodiment, the method according to invention is disclosed, wherein the subject is a mammal, preferably a human.

In an embodiment, the method according to the invention is disclosed, wherein said relationship is between two biomarkers such as pH vs. NH₄⁺, TA vs NH₄⁺, TB vs. NH₄⁺, BB vs NH₄⁺, HCO₃⁻ vs. NH₄⁺, pH vs. HCO₃⁻, and pH vs. TA.

In an embodiment, the method according to the invention is disclosed, wherein said relationship is between two biomarkers NH₄⁺ vs. pH.

In an embodiment of the invention, preferably said two biomarkers are pH vs. NH₄⁺. Surprisingly strong data support these two biomarkers as exceedingly good tools that are able to identify acid retention and SA as a clinically relevant measure that can be easily detected in simple urine collections from CKD patients. These data are presented in FIGS. 2-5.

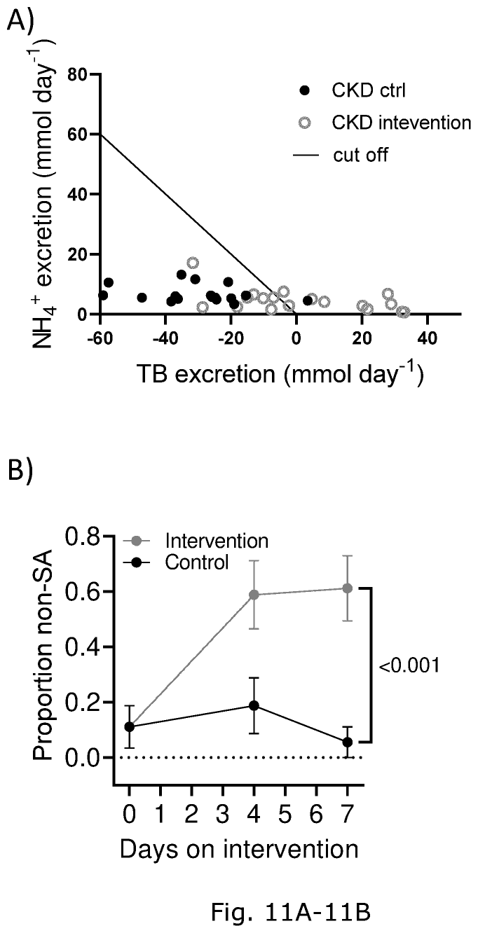

In an embodiment, said two biomarkers are TA vs NH₄⁺, as shown in FIGS. 8-9 In an embodiment, preferably said two biomarkers are TB vs. NH₄⁺, as shown in FIGS. 10 and 11.

In an embodiment, preferably said two biomarkers are BB vs NH₄⁺, as shown in FIGS. 13A and 14.

In an embodiment, preferably said two biomarkers are HCO₃-vs. NH₄⁺, as shown in FIGS. 13B and 15.

In an embodiment, the method according to the invention is disclosed, wherein the score is a binary or continuous score. In an embodiment, a method according to the invention is disclosed, wherein said score is positive or negative.

In an embodiment of the invention, said score being positive or negative is obtained by calculating one or more cut-off lines.

In an embodiment of the invention, said method is disclosed, wherein scores that are negative compared to the calculated cut-off line encompass subjects that are considered at risk of having subclinical acidosis and/or subjects that have subclinical acidosis.

In an embodiment of the invention, said method is disclosed, wherein scores that are positive compared to the calculated cut-off line encompass subjects that are considered not at risk of having subclinical acidosis and/or subjects that are considered not having subclinical acidosis.

In another embodiment, said cut-off line is algorithmic or linear. In a preferred embodiment, said cut-off line is a non-linear. In an even more preferred embodiment, said non-linear cut-off equation is scoring concept 2.

In an embodiment of the invention, said cut-off line is selected from one or more of scoring concept 1, scoring concept 2, scoring concept 3, scoring concept 4, scoring concept 5 or scoring concept 6. Preferably, said score is selected from scoring concept 2.

In an embodiment, said method according to the invention is disclosed, wherein the level of NH₄⁺, TA, BB, TB and/or HCO₃⁻ is the concentration of NH₄⁺, TA, BB, TB and/or HCO₃⁻.

In an embodiment of the invention, said method is disclosed, wherein the level of NH₄⁺, TA and TB is the excretion of NH₄⁺, TA and TB.

In an embodiment, said method according to the invention is disclosed, wherein the level of NH₄⁺ is the NH₄⁺/creatinine ratio, the level of TA is the TA/creatinine ratio and wherein the level of BB is the BB/creatinine ratio.

In an embodiment, said method according to the invention is disclosed, wherein the level of NH₄⁺ is the NH₄⁺/creatinine ratio and wherein the level of HCO₃⁻ is the HCO₃⁻/creatinine ratio.

In an embodiment, said method according to the invention is disclosed, wherein the score is an arithmetic relation correlating the levels of biomarkers.

In a preferred embodiment, the present invention relates to a method according to the invention, wherein the score is an arithmetic relation correlating the levels of at least two biomarkers.

In a further preferred embodiment, the present invention relates to a method according to the invention, wherein the score is an arithmetic relation correlating the levels of two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻.

In a more preferred embodiment, the present invention relates to a method according to the invention, wherein the score is an arithmetic relation correlating the levels of the biomarkers NH₄⁺ vs. pH.

In an embodiment of the invention, the present invention relates to a method according to the invention, wherein the score is an arithmetic relation determined by NH₄⁺≥−15*pHᵤ+97.5. The formula is scoring concept 1.

In an embodiment of the invention, the present invention relates to a method according to the invention, wherein the score is an arithmetic relation determined by (log ([NH₄⁺]ᵤ)·(pHᵤ³))/10. The formula is scoring concept 2 or the variant of scoring concept 2 called “AB_score”. Although this embodiment has been described in connection with the specified embodiment, it should not be construed as being in any way limited to the present aspect, but can be applied to aspects 1 to 5 as used herein.

In an embodiment of the invention, the present invention relates to a method according to the invention, wherein the score is an arithmetic relation determined by log [NH₄⁺]ᵤ*log (([TA]ᵤ*−1)+28). The formula is scoring concept 3.

In an embodiment of the invention, the present invention relates to a method according to the invention, wherein the score is an arithmetic relation determined by NH₄⁺ excretion≥−1*TB excretion+0. The formula is scoring concept 4.

In an embodiment of the invention, the present invention relates to a method according to the invention, wherein the score is an arithmetic relation determined by [NH₄⁺]ᵤ/[creatinine]ᵤ≥−2.5*[BB]ᵤ/[Creatinine]ᵤ+0. The formula is scoring concept 5.

In an embodiment of the invention, the present invention relates to a method according to the invention, wherein the score is an arithmetic relation determined by [NH₄⁺]ᵤ/[creatinine]ᵤ≥−150*[HCO₃⁻]ᵤ/[Creatinine]ᵤ+0. The formula is scoring concept 6.

A second aspect of the invention relates to a method for monitoring the development of subclinical acidosis (SA) in a subject suffering from chronic kidney disease (CKD) or having acid retention, the method comprising
determining a first score based on a relationship between the levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻, said biomarkers being measured or sensed in a first urine sample from the subject;
determining a second score based on a relationship between the levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻, said biomarkers being measured or sensed in a second urine sample from the subject, wherein the second sample has been obtained at a later time point than the first sample;
comparing corresponding scores in the first and second sample;
wherein
   a negative score in the second sample compared to a positive score in the first sample is indicative of development of subclinical acidosis;
   a positive score in the second sample compared to positive score in the first sample is indicative of no development of subclinical acidosis;
   a negative score in the second sample compared to a negative score in the first sample is indicative of maintained subclinical acidosis;
   a positive score in the second sample compared to negative score in the first sample is indicative of improved/lessening of subclinical acidosis.

In an embodiment a method for monitoring the development of subclinical acidosis (SA) or acid retention in a subject suffering from chronic kidney disease (CKD) is disclosed, the method comprising
determining a first score based on the levels of at least two biomarkers selected from the group consisting of NH₄⁺, TA, TB, BB, pH and HCO₃⁻, said biomarkers being measured or calculated in a first urine sample from the subject;
determining a second score based on the levels of at least two biomarkers selected from the group consisting of NH₄⁺, TA, TB, BB, PH and HCO₃⁻, said biomarkers being measured or calculated in a second urine sample from the subject, wherein the second sample has been obtained at a later time point than the first sample;
comparing corresponding scores in the first and second sample;
wherein
   a negative score in the second sample compared to a positive score in the first sample is indicative of development of subclinical acidosis or acid retention;
   a positive score in the second sample compared to positive score in the first sample is indicative of no development of subclinical acidosis or acid retention;
   a negative score in the second sample compared to a negative score in the first sample is indicative of maintained subclinical acidosis or acid retention;
   a positive score in the second sample compared to negative score in the first sample is indicative of improved subclinical acidosis or acid retention.

In an embodiment of the invention, said subject
has subclinical acidosis (SA), if said score is negative; and
does not have subclinial acidosis (non-SA), if said score is positive.

In an embodiment of the invention, said first urine sample is a 24 h urine collection and said second urine sample is a 24 h urine collection.

In another embodiment, said first urine sample is a spot urine sample and said second urine sample is a spot urine sample.

In an embodiment of the invention, said subject is a mammal, preferably a human.

In an embodiment, the score is a binary or continous score. In another embodiment, said score is binary. In yet another embodiment, said score is continous. In a method according to the invention, said score is positive or negative.

In an embodiment of the invention, said score being positive or negative is obtained by calculating one or more cut-off lines.

In an embodiment of the invention, said score being positive or negative is obtained by calculating one or more cut-off lines.

In an embodiment of the invention, said method is disclosed, wherein scores that are negative compared to the calculated cut-off line encompass subjects that are considered at risk of having subclinical acidosis and/or subjects that have subclinical acidosis.

In an embodiment of the invention, said method is disclosed, wherein scores that are positive compared to the calculated cut-off line encompass subjects that are considered not at risk of having subclinical acidosis and/or subjects that are considered not having subclinical acidosis.

In another embodiment, said cut-off line is algorithmic or linear. In a preferred embodiment, said cut-off line is a non-linear. In an even more preferred embodiment, said non-linear cut-off equation is scoring concept 2.

In an embodiment of the invention, said cut-off line is selected from one or more of scoring concept 1, scoring concept 2, scoring concept 3, scoring concept 4, scoring concept 5 or scoring concept 6. Preferably, said score is selected from scoring concept 2.

In an embodiment of the invention, measuring or calculating in a first or second urine sample from a subject, the level of at least tree biomarkers are selected from the group consisting of NH₄⁺, TA, TB, BB, pH and HCO₃⁻.

In another embodiment of the invention, measuring or calculating in a first or second urine sample from a subject, the level of at least four biomarkers are selected from the group consisting of NH₄⁺, TA, TB, BB, PH and HCO₃⁻.

In an embodiment, the level of NH₄⁺, TA, BB, TB and/or HCO₃⁻ is the concentration of NH₄⁺, TA, BB, TB and/or HCO₃⁻.

In an embodiment, the level of NH₄⁺, TA and TB is the excretion of NH₄⁺, TA and TB.

In an embodiment of the invention, wherein the level of NH₄⁺ is the NH₄⁺/creatinine ratio, the level of TA is the TA/creatinine ratio and wherein the level of BB is the BB/creatinine ratio.

In an embodiment, wherein the level of NH₄⁺ is the NH₄⁺/creatinine ratio and wherein the level of HCO₃⁻ is the HCO₃⁻/creatinine ratio.

In a further embodiment, said relationship is between said two biomarkers NH₄⁺ vs. pH in said first urine sample and said second urine sample.

In an embodiment of the invention, the score is an arithmetic relation correlating the levels of biomarkers.

In an embodiment, said two biomarkers in said first urine sample and in said second urine sample are the same two biomarkers.

In an embodiment of the invention, said subject is suffering from chronic kidney disease (CKD) selected from grade CKD grade G2, such as G3a, such as G3b, such as G3, such as G4, such as G5, such as G2-G3, such as G2-G4, such as G3-G4, such as G4-G5, such as G3a-G3b, such as G2-G3a, such as G2-G3b, such as G3a-G4, such as G3b-G4, such as G3-G5. Preferably, said subject has CKD G2 or G3, or even more preferably G2-G3.

In an embodiment, said method according to the invention is disclosed, wherein a treatment of subclinical acidosis has taken place between the sampling of the first sample and the second sample.

In an embodiment of the invention, said treatment is selected from acid-reducing dietary regimes, base supplementation, pharmacological treatment or gastrointestinal proton chelators.

In a preferred embodiment of the invention, said treatment is selected from acid-reducing dietary regimes.

A third aspect of the invention relates to, a method for determining the effect of a treatment protocol against subclinical acidosis (SA) or acid retention for a subject suffering from chronic kidney disease (CKD), the method comprising
determining a first score based on a relationship between the levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻, said biomarkers being measured or sensed in a first urine sample from the subject;
determining a second score based on a relationship between the levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻, said biomarkers being measured or sensed in a second urine sample from the subject, wherein the second sample has been obtained at a later time point than the first sample;
   wherein the treatment protocol has been initiated or completed before the sampling of the first sample or initiated, continued or completed between the sampling of the first and second sample,
comparing scores of the first sample and the second sample; wherein
   a negative score in the second sample compared to a positive score in the first sample is indicative of the treatment protocol not being effective against subclinical acidosis or acid retention;
   a negative score in the second sample compared to a negative score in the first sample is indicative of the treatment protocol not being effective against subclinical acidosis or acid retention;
   a positive score in the second sample compared to negative score in the first sample is indicative of the treatment protocol being effective against subclinical acidosis or acid retention.

An embodiment of the invention relates to a method for determining the effect of a treatment protocol against subclinical acidosis (SA) or acid retention for a subject suffering from chronic kidney disease (CKD), the method comprising
determining a first score based on the levels of at least two biomarkers selected from the group consisting of NH₄⁺, TA, TB, BB, PH and HCO₃⁻, said biomarkers being measured or calculated in a first urine sample from the subject;
determining a second score based on the levels of at least two biomarkers selected from the group consisting of NH₄⁺, TA, TB, BB, PH and HCO₃⁻, said biomarkers being measured or calculated in a second urine sample from the subject, wherein the second sample has been obtained at a later time point than the first sample;
   wherein the treatment protocol has been initiated or completed before the sampling of the first sample or initiated, continued or completed between the sampling of the first and second sample,
comparing scores of the first sample and the second sample; wherein
   a negative score in the second sample compared to a positive score in the first sample is indicative of the treatment protocol not being effective against subclinical acidosis or acid retention;
   a negative score in the second sample compared to a negative score in the first sample is indicative of the treatment protocol not being effective against subclinical acidosis or acid retention;
   a positive score in the second sample compared to negative score in the first sample is indicative of the treatment protocol being effective against subclinical acidosis or acid retention.

In an embodiment of the invention, said subject
has subclinical acidosis (SA), if said score is negative; and
does not have subclinical acidosis (non-SA), if said score is positive.

In an embodiment of the invention, said first urine sample is a 24 h urine collection and said second urine sample is a 24 h urine collection.

In another embodiment of the invention, said first urine sample is a spot urine sample and said second urine sample is a spot urine sample.

In an embodiment, the subject is a mammal, preferably a human.

In an embodiment, the score is a binary or continuous score. In another embodiment, said score is binary. In yet another embodiment, said score is continuous. In a method according to the invention, said score is positive or negative.

In an embodiment of the invention, said score being positive or negative is obtained by calculating one or more cut-off lines.

In an embodiment of the invention, said method is disclosed, wherein scores that are negative compared to the calculated cut-off line encompass subjects that are considered at risk of having subclinical acidosis and/or subjects that have subclinical acidosis.

In an embodiment of the invention, said method is disclosed, wherein scores that are positive compared to the calculated cut-off line encompass subjects that are considered not at risk of having subclinical acidosis and/or subjects that are considered not having subclinical acidosis.

In another embodiment, said cut-off line is algorithmic or linear. In a preferred embodiment, said cut-off line is a non-linear. In an even more preferred embodiment, said non-linear cut-off equation is scoring concept 2.

In an embodiment of the invention, said cut-off line is selected from one or more of scoring concept 1, scoring concept 2, scoring concept 3, scoring concept 4, scoring concept 5 or scoring concept 6. Preferably, said score is selected from scoring concept 2.

In an embodiment of the invention, measuring or calculating in a first or second urine sample from a subject, the level of at least tree biomarkers are selected from the group consisting of NH₄⁺, TA, TB, BB, pH and HCO₃⁻.

In another embodiment of the invention, measuring or calculating in a first or second urine sample from a subject, the level of at least four biomarkers are selected from the group consisting of NH₄⁺, TA, TB, BB, PH and HCO₃⁻.

In an embodiment, the level of NH₄⁺, TA, BB, TB and/or HCO₃⁻ is the concentration of NH₄⁺, TA, BB, TB and/or HCO₃.

In an embodiment, the level of NH₄⁺, TA and TB is the excretion of NH₄⁺, TA and TB.

In a further embodiment of the invention, the level of NH₄⁺ is the NH₄⁺/creatinine ratio, the level of TA is the TA/creatinine ratio and the level of BB is the BB/creatinine ratio.

In yet an embodiment, the level of NH₄⁺ is the NH₄⁺/creatinine ratio and the level of HCO₃⁻ is the HCO₃⁻/creatinine ratio.

In an embodiment, said relationship is between said two biomarkers NH₄⁺ vs. pH in said first urine sample and said second urine sample.

In an embodiment of the invention, the score is an arithmetic relation correlating the levels of biomarkers.

In an embodiment, said two biomarkers in said first urine sample and in said second urine sample are the same two biomarkers.

In an embodiment of the invention, said subject is suffering from chronic kidney disease (CKD) selected from grade CKD grade G2, such as G3a, such as G3b, such as G3, such as G4, such as G5, such as G2-G3, such as G2-G4, such as G3-G4, such as G4-G5, such as G3a-G3b, such as G2-G3a, such as G2-G3b, such as G3a-G4, such as G3b-G4, such as G3-G5. Preferably, said subject has CKD G2 or G3, or even more preferably G2-G3.

In an embodiment, the method according to the invention is disclosed, wherein the treatment is acid-reducing dietary regimes, base supplementation, pharmacological treatment or gastrointestinal proton chelators.

In an embodiment, the method according to the invention is disclosed, wherein the treatment is acid-reducing dietary regimes.

A fourth aspect of the invention relates to use of urine sample levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻ as biomarkers for determining the risk of a subject of having subclinical acidosis or having acid retention.

An embodiment of the invention relates to the use of urine sample levels of at least two biomarkers selected from the group consisting of NH₄⁺, TA, TB, BB, pH and HCO₃⁻ as biomarkers for determining the risk of a subject of having subclinical acidosis.

Yet another embodiment of the invention relates to the use of urine sample levels of at least two biomarkers selected from the group consisting of NH₄⁺, TA, TB, BB, pH and HCO₃⁻ as biomarkers for determining the risk of a subject of having acid retention.

A fifth aspect of the invention relates to use of urine sample levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻ as biomarkers for determining the risk of a subject of having subclinical acidosis or having acid retention to predict serious clinical event-free survival of said subject.

Yet another embodiment of the invention relates to the use of urine sample levels of at least two biomarkers selected from the group consisting of NH₄⁺, TA, TB, BB, pH and HCO₃⁻; as biomarkers for determining the risk of a subject of having subclinical acidosis to predict serious clinical event-free survival of said subject.

Yet a further embodiment of the invention relates to the use of urine sample levels of at least two biomarkers selected from the group consisting of NH₄⁺, TA, TB, BB, pH and HCO₃⁻ as biomarkers for determining the risk of a subject of having acid retention to predict serious clinical event-free survival of said subject.

It should be noted that embodiments and features described in the context of one of the aspects of the present invention also apply to the other aspects of the invention. Thus, for example the embodiments/claims relating to the method of the invention also apply to the embodiments relating to the use-embodiments/claims. Hence, individual features mentioned in different claims, may possibly be advantageously combined, and the mentioning of these features in different claims does not exclude that a combination of features is not possible and advantageous.

Although the present invention has been described in connection with the specified embodiments, it should not be construed as being in any way limited to the presented examples. The scope of the present invention is to be interpreted in the light of the accompanying claim set. In the context of the claims, the terms “comprising” or “comprises” do not exclude other possible elements or steps. Also, the mentioning of references such as “a” or “an” etc. should not be construed as excluding a plurality.

All patent and non-patent references cited in the present application, are hereby incorporated by reference in their entirety.

The invention will now be described in further details in the following non-limiting examples.

### EXAMPLES—SHORT OVERVIEW

Example 1 Study design, participants and methods used for data collection and calculations

Example 2 The absent physiological response to increase acid excretion in CKD patients

Example 3 The urinary [NH₄⁺] and urinary pH relationship

Example 4 The urinary [NH₄⁺] and urinary [TA] relationship

Example 5 The urinary NH₄⁺ excretion and the urinary total base excretion rate relationship

Example 6 The relative (normalized to creatinine) NH₄⁺ and base buffer/bicarbonate excretion relationship

Example 7 Determining a urinary acid/base score (AB_score)

### Example 1—Study Design, Participants and Methods Used for Data Collection and Calculations

### Materials and Methods

### Study Design and Participants

Samples: Human urine samples were obtained from two different biobanks. One cohort consisted of 24 h urine collections from 40 healthy controls and 82 CKD patients (grade 3-4). These samples were initially collected at the Aarhus University Hospital from 2011-2013. The second cohort consisted of 24 h urine collections from 18 CKD (grade 3-4) patients enrolled in a randomized controlled crossover study investigating a dietary intervention. These samples were collected in 2017. We refer to the study numbers NCT03052582 (clinicaltrials.gov) and RenVas, which is EudraCT number 2010-023979-25, clinical trial number NCT01380717.

For examples 3-7, the below tables show cohort demographic and clinical data for the CKD patients and controls used. Please note that for the dietary intervention studies (Examples 3B, 3D, 5B), patient cohort demographic and clinical data from the controls are shown in Table 3.

TABLE 2
Main patient cohort demographic and clinical data.

| | Controls, n = 25 | CKD, n = 82 |
|---|---|---|
| | (mean) | (mean) |
| Age - years (SD) | 62 (12) | 65 (13) |
| Sex (female) - % | 21% | 27% |
| BMI - kg/m² (SD) | 24 (3) | 27 (4) |
| mGFR - mL/min/1.73 m² (SD) | 97 (23) | 36 (15) |
| UAC - mg/mmol [IQR] | 0 [0-0.2] | 7 [0-76] |
| tCO² - mM (SD) | 28 (2) | 28 (4) |

TABLE 3
Intervention (dynamic response) patient
cohort demographic and clinical data

| Number of CKD patients | n = 18 (mean) |
|---|---|
| Age - years (SD) | 53 (13) |
| Sex (female) - % | 50% |
| BMI - kg/m² (SD) | 27 (2.7) |
| eGFR - mL/min/1.73 m² (SD) | 28 (9) |
| tCO² - mM baseline (SD) | 23.4 (3.2) |

### Methods Used for Data Collection and Calculation:

### Urine Analysis:

Urine ammonium ([NH₄⁺]ᵤ) was measured using an Orion™ High-Performance Ammonia Ion-Selective Electrode (Thermo Scientific, Cat. No. 9512HPBNWP) with the use of Ammonia pH-adjusting Ionic Strength Adjuster (Thermo Scientific, Cat. No. 951211).

Urine pH ([pH]ᵤ) was measured with a pH electrode (Metrohm).

Urine titratable acids (TAᵤ) were measured by titration using the method of Chan (described in Chan J C M, Clin. Biochem. 5, 94-98 (1972) with the use of an automated titrator system (Eco Titrator, Metrohm). In short, an equal volume of 1M HCl was added to urine samples. Subsequently, the samples were shortly boiled (1 min) and when cooled to room temperature whereafter they were titrated to pH 7.4 by the addition of 1M NaOH. The difference between NaOH added to samples and pure water controls were used to calculate the concentration of urinary TA.

Urine bicarbonate ([HCO₃⁻]ᵤ) was measured utilizing an infra-red CO₂-sensor-based system (CO₂ meter GM70, Vaisala). In short, HCO₃; was released from the liquid phase as CO₂ to the gas phase by the addition of a surplus amount of HCl. The increase in CO₂ in the gas phase is then detected with an infra-red CO₂-sensor. Based on readouts from a known HCO₃⁻ standard curve the initial sample [HCO₃⁻] was back-calculated.

TABLE 4
Formulas for calculation of parameters used in urine analysis

| | Formula for |
|---|---|
| Parameters | calculation of parameters |
| Urine base buffers | BB = TA * −1 |
| (BB or U_{BB}) | |
| Urine total base (TB or U_{TB}) | TB = ([BB] + [HCO₃⁻]) |
| Urine total base excreted | Urine total base excreted = TBᵤ * |
| | 24 h urine volume. |
| Urine NH₄⁺ excretion | Urine NH₄⁺ excretion = [NH₄⁺] * |
| | 24 h urine volume. |
| ΔmGFR | GFR measured at the first clinical |
| | visit subtracted by measured GFR |
| | at the second clinical visit |
| | (18 months later). |

### Example 2—The Absent Physiological Response to Increase Acid Excretion in CKD Patients

### Aim of Study

To evaluate the physiological response to increased acid excretion in CKD patients compared to healthy control patients.

### Materials and Methods

See also example 1.

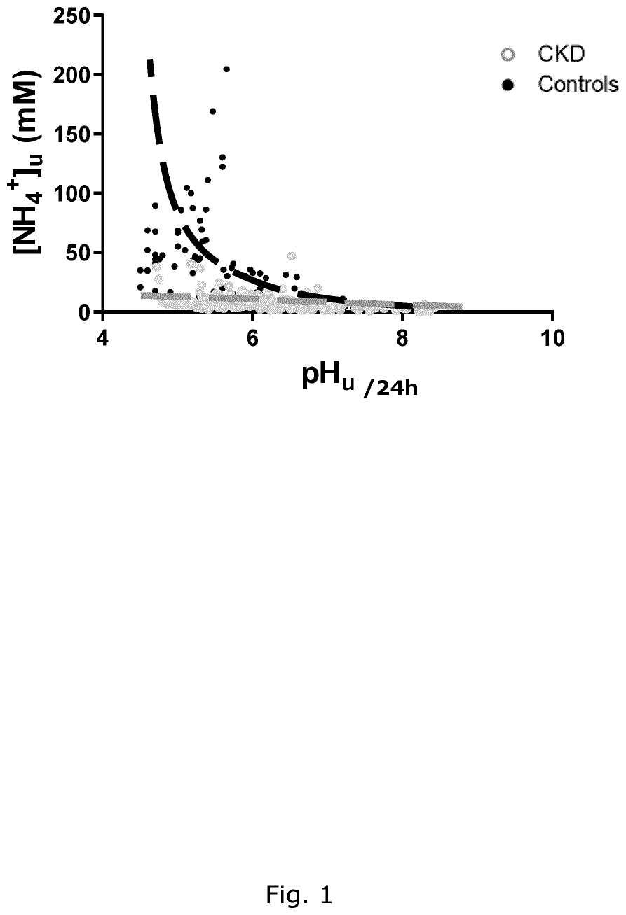

The concentration of NH₄⁺ and the pH in 24 h urine samples from CKD patients and healthy controls were measured. FIG. 1 shows measurements of urine [NH₄⁺]ᵤ plotted against pH in 24 h urine samples obtained from 214 CKD patients and 82 healthy controls.

### Results

FIG. 1: The figure demonstrated the well-known and normal responses from healthy controls to an augmented need for acid excretion, which is seen as a reduction in urine pH and a concurrent increase in urine [NH₄⁺] (black dots and black curve). Contrary, CKD patients fail to increase urinary NH₄⁺ when urinary protonation increases (when there is a fall in pHᵤ) i.e. when there is a physiological need for acid excretion. This is seen as a fall in pHᵤ (grey dots and grey line). This well known response is also shown in Chan J C M, Clin. Biochem. 5, 94-98 (1972). Data presented in FIG. 1 also stem from data shown in the paper from Elinton J R et al. p. 554-575, American Journal of Medicin, Clinical Studies, October 1960 and from the paper from Schwartz W B et al, “On the mechanism of acidosis in chronic renal disease”, (submitted 1958/accepted Sep. 11, 1958).

### Conclusion

This analysis reveals that CKD patients are unable to increase urinary [NH₄⁺] during acidosis, which is otherwise the normal physiological response for healthy humans. Accordingly, FIG. 1 demonstrates that there is an absence of a normal physiological response to increased acid excretion in CKD patients.

### Example 3 Explores the Urinary [NH₄⁺] and Urinary pH Relationship—Examples 3A, 3B, 3C and 3D

### Example 3A—Urine NH₄⁺ vs. Urinary pH Quantify a Systemic Acid Overload Scoring Concept 1

### Aim of Study

To evaluate the relevance of the relationship between urine NH₄⁺ and urine pH in CKD patients and healthy controls.

Further, to evaluate the relevance of a subdivision of CKD patients into SA or non-SA groups using a linear cut-off line to identify patients with progression of kidney function loss and to study this relevance during 7 years of clinical follow-up.

### Materials and Methods

See also example 1.

Scoring concept 1 was used for this example being a linear cut-off line.

Based on the [NH₄⁺]ᵤ and pH relationship and a linear cut-off between SA and non-SA.

non-SA when NH₄⁺≥−15*pHᵤ+97.5

or non-SA when pHᵤ≥6.5

Thus, in FIG. 2A, [NH₄⁺]ᵤ (mM) was plotted as a function of urine pH in CKD patients and healthy controls. The inclusion criteria for the selection of CKD patients comprised a normal systemic acid/base status, i.e. no apparent metabolic acidosis. “No apparent metabolic acidosis” is defined herein as the standard HCO₃ concentration ≥22 mmol/l or total CO₂ under 22 mmol/l). In other words, these patients are not considered to have apparent metabolic acidosis and are therefore CKD patients that comprise a normal systemic acid/base status.

The number of observations: CKD 82 and control 25 urine samples.

Based on the identification that a large number of CKD patient points have very low urine [NH₄⁺] and very acidic urine pH, a linear cut-off line [(pHᵤ=0), ([NH₄⁺]ᵤ=30]); [(pHᵤ=6.5), ([NH₄⁺]ᵤ=0)] was added to FIG. 2A, as calculated based on scoring concept 1. This cut-off line was surprisingly able to fully separate the CKD patients with apparent acid retention from those CKD patients with similar urine [NH₄⁺] but at more alkaline urine i.e. a urine with higher pH. The latter group was much more alike to the urine measurements of healthy controls (black).

In FIG. 2B, data from 82 CKD patients are shown where kidney function loss (AmGFR) was plotted against the SA-scoring identified from the data in FIG. 2A. In all CKD patients (SA or Non-SA patients), the progression of kidney function loss (AmGFR) was followed for 1.5 years i.e. 18 months. A negative value of ΔmGFR indicates further kidney function loss.

TABLE 5
Number of patients used in FIG. 2B

| Division of CKD patients into SA score | SA | Non-SA |
|---|---|---|
| Number of patients (N)) | 50 | 32 |

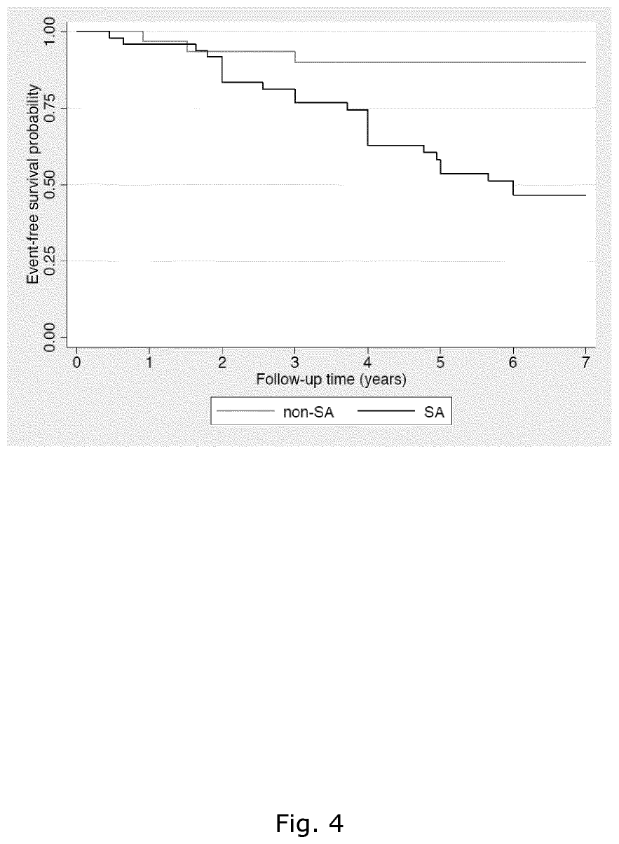

In FIG. 4, data from FIG. 2A is plotted as event-free survival probability of SA and non-SA scored CKD patients (urine NH₄⁺ vs. urinary pH) in a Kaplan-Meyer plot that displays the probability of serious clinical event-free survival (further 50% reduction in GFR, entering dialysis or kidney transplantation) of the SA and non-SA scored CKD patients measured each year up to 7 years. Patients scored as SA or non-SA based on urinary [NH₄⁺] pH relationship and the linear cut-off displayed in FIG. 2A.

### Results—FIGS. 2A, 2B and 4

FIG. 2A: A large number of CKD patient points have very low urine [NH₄⁺] and very acidic urine pH. This is likely to indicate that these CKD patients suffer from significant acid retention that is not apparent in the acid/base status of a blood sample i.e. CKD patients that meet the standard HCO₃ concentration >22 mmol/l or total CO² under 22 mmol/l). In order to quantify this observation, the inventors added a linear cut-off line [(pHᵤ=0), ([NH₄⁺]ᵤ=30]); [(pHᵤ=6.5), ([NH₄⁺]ᵤ=0)], as shown in FIG. 2A. This linear line was hereafter used to fully separate the CKD patients with apparent acid retention from those CKD patients with similar urine [NH₄⁺] but at more alkaline urine (i.e. a pH to the right in the figure). This latter group of CKD patients was much more alike to the urine measurements of controls (black). This permitted a subdivision of CKD patients with a urine analysis indicating acid retention (left of line) from a group that was apparently non-acid retaining (right of line). These patients were scored to have SA (subclinical acidosis) or non-SA (no subclinical acidosis), respectively. The data in FIG. 2A showed a surprising difference between the CKD and the control group.

FIG. 2B: The scored SA or non-SA patient groups identified and calculated from the data presented in FIG. 2A were plotted against kidney function loss (AmGFR) after 18 months. Accordingly, FIG. 2B show data from 82 CKD patients which suggests that those CKD patients categorized with SA differed markedly and surprisingly from those categorized as being non-SA in the key clinical hallmark of CKD progression being a reduction of GFR (kidney function loss) after 18 months.

FIG. 4: The scored SA or non-SA patient groups identified and calculated from the data presented in FIG. 2A were plotted in a Kaplan-Meyer plot displaying the probability of serious clinical event-free survival (further 50% reduction in GFR, entering dialysis or kidney transplantation) of the SA and non-SA scored CKD patients over the course of 7 years. The results are summarized in Table 6.

Table 6: Hazard ratios calculated by a cox proportional hazards model based on the linear cut-off line displayed in FIG. 2A (Kaplan-Meyer FIG. 4). Please note that patients scored SA has a 6.1 times higher risk of meeting a serious clinical event than non-SA scored patients. Even after adjustment for key competing clinical risk-factors (Age, GFR, BMI, urine albumin creatinine ratio, tCO₂ and blood pressure all at the time of scoring) SA scored patients has a 2.6 timer high risk of experiencing a serious clinical event.

Hazard ratios (95% CI)

| | SA score (SA yes/no) | SA score (per unit) |
|---|---|---|
| Unadjusted | 6.1 (1.8 to 20.4) | 0.93 (0.89 to 0.97) |
| Adjusted | 2.6 (0.7 to 9.8) | 0.97 (0.92 to 1.02) |

Hazard ratio adjusted for: Age (years), sex, BMI (kg/m²), baseline eGFR (mL/min/1.73 m²), urine albumin creatinine ratio (mg/g), tCO₂ (mM), and systolic blood pressure (mmHg).

### Conclusion

FIG. 2A surprisingly demonstrates that it is possible to divide CKD patients into groups having SA or non-SA, respectively by way of the suggested linear cut-off line between the score of SA or non-SA when using the relationship between urine NH₄⁺ and urine pH.

FIG. 2B relates to the development of GFR among CKD patients that scored SA or non-SA. Thus, FIG. 2B surprisingly demonstrates that the patients with SA showed accelerated loss of kidney function within a period of 18 M compared to the non-SA group, whereas the non-SA group showed stable kidney function within a period of 18M.

FIG. 4 surprisingly demonstrates that patients who scored SA has a 6.1 times higher risk of meeting a serious clinical event than non-SA scored patients when followed for 7 years. Even after adjustment for key competing clinical risk-factors, SA scored patients have a 2.6 timer high risk of experiencing a serious clinical event.

Thus, the inventors have surprisingly identified that the relationship between urinary NH₄⁺ and urinary pH in CKD-patients can be used to identify CKD-patients, by use of the scoring concept 1, that are at high risk of developing kidney function loss (AmGFR) within e.g. 18 months and/or meeting a serious clinical event when followed for 7 years. By early identification of such risks, interventions to prevent kidney function loss can be initiated to avoid said risks.

### Example 3B—Dynamic Response: Dietary Intervention on the Relationship Between Urinary NH₄⁺ Concentration and Urinary pH in CKD Patients—Scoring Concept 1

### Aim

To evaluate the relevance of the SA/non-SA score for CKD patients when subjected to a dietary intervention that is known to reduce the systemic acid load and thus reduce the risk of acid overload in CKD patients.

### Materials and Methods

See example 1 and 3A.

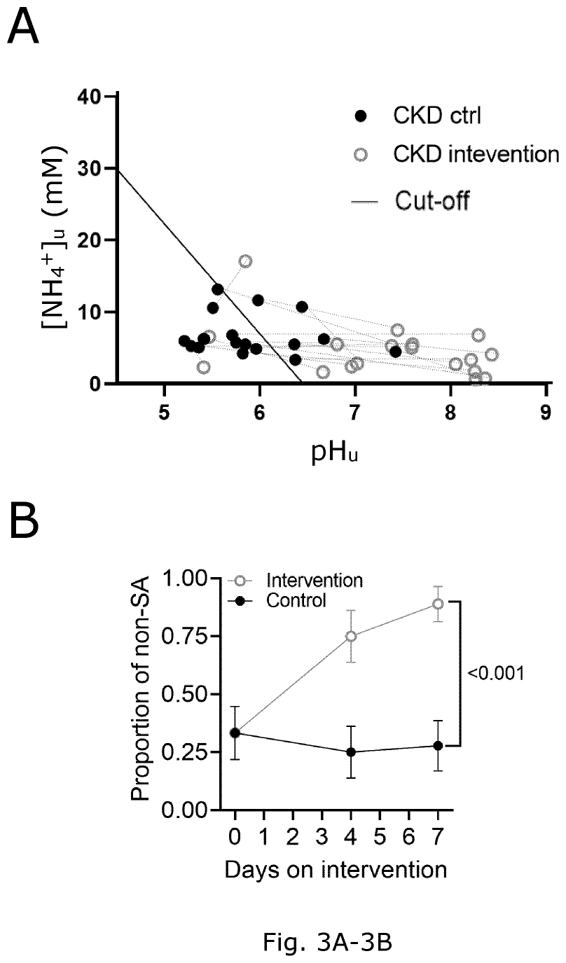

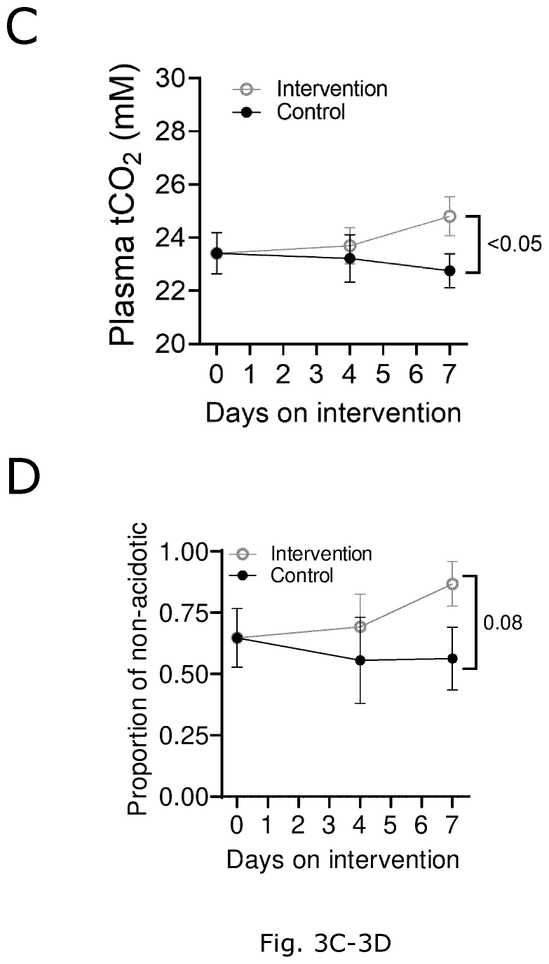

FIG. 3A: To further assess the possible value of the suggested SA/non SA groups, the inventors analyzed 18 CKD patients before and after a short-term acid-reducing dietary intervention (New Nordic Renal Diet, NNRD) i.e. 1 week (seven days) intervention. The NNRD is known to reduce the systemic acid load and thus reduce the risk of acid overload in CKD patients. Thus, the urinary NH₄⁺ concentration and urinary pH were measured in all 18 CKD patients before and after the short-term dietary intervention. Two measurements from each patient are linked with thin lines in FIG. 3A. In order to quantify the observations, the inventors added a linear “cut-off” line [(pHᵤ=0), ([NH₄⁺]ᵤ=30]); [(pHᵤ=6.5), ([NH₄⁺]ᵤ=0)], as shown in FIG. 3A. The presented data were obtained from 24 h urine collections. Table 3 shows the intervention (dynamic response) patient cohort demographic and clinical data. Please note that the “cut-off” line used in FIG. 3A is used to assess the value of the SA/non-SA groups that have been provided using the scoring concepts calculations.

FIG. 3B: The urinary NH₄⁺ concentration and urinary pH were measured in all 18 CKD patients before intervention and at day four and seven of the intervention period. The proportion of non-SA patients was registered, as calculated based on the cut-off value. The proportion value on SA were plotted against the days of intervention.

FIG. 3C: The total plasma CO₂ (tCO₂) as a measure of systemic acid/base status was also measured on all 18 CKD patients before intervention and at day 4 and seven of the intervention period.

FIG. 3D:

The urinary NH₄⁺ concentration and urinary pH were measured in all 18 CKD patients before intervention and at day four and seven of the intervention period. The proportion of non-acidotic patients was registered, as calculated based on the cut-off value. The proportion value of non-acidotic were plotted against the days of intervention.

### Results—FIGS. 3A, 3B, 3C and 3D

FIG. 3A: FIG. 3A shows the relationship between urinary NH₄⁺ concentration and urinary pH with the suggested linear cut-off line as defined in example 3 between the score of SA or non-SA. Black dots are baseline and grey dots after 1-week dietary intervention (NNRD) where two measurements from each patient are linked with thin lines. The black dots represents CDK ctrl; i.e. CKD levels before dietary intervention (day zero) and the grey dots represents CKD intervention i.e. CKD levels after dietary intervention (day seven).

It is surprisingly shown that the dietary intervention with the NNRD after just seven days shifts a significant number of CKD patients to the right side of the cut-off line. This means that the results show that CKD patients scored SA, become non-SA after only seven days on the NNRD. These results were quantified and are shown in FIG. 3B.

FIG. 3B: The fraction of CKD patients that scored non-SA increased highly surprisingly from ~33% to ~90% after a week on the NNRD.

FIG. 3C: The same CKD patients undergoing dietary intervention as discussed under FIGS. 3A and 3B were investigated with regard to their total plasma CO₂ level, as a measure of systemic acid/base status. Surprisingly, it was shown that the proportion of non-SA-scored patients increased already at day 4 (FIG. 3C). Change in systemic acid/base status was less prominent when compared to FIG. 3B and only significant after a full week of intervention.

FIG. 3D: The fraction of non-acidotic patients that scored non-acidotic increased highly surprisingly from ~65% to ~87% after a week on the NNRD.

### Conclusion

Thus, surprisingly, the urine analysis appears a very sensitive measure to monitor a dynamic effect of a short-term acid-reducing intervention such as the NNRD as compared to the changes in blood acid/base measures (FIGS. 3A+2B compared to FIG. 3C or 3D). Surprisingly, the fraction of CKD patients that scored non-SA, based on a linear cut-off line, increased from about 30% to ~90% after a week of dietary intervention. This is supported by the fraction of fully acidotic CKD patients that scored non-acidotic, based on a linear cut-off line, which increased from about 65% to ~87% after a week of dietary intervention.

### Example 3C—Urine NH₄⁺ vs. Urinary pH Quantifies a Systemic Acid Overload-Scoring Concept 2

### Aim of Study

To evaluate the relevance of the relationship between urine NH₄⁺ and urine pH in CKD patients and healthy controls.

Further, to evaluate the relevance of a subdivision of CKD patients into SA or non-SA groups using a non-linear cut-off equation to identify patients with progression of kidney function loss and to study this relevance for the duration of 7 years.

### Materials and Methods

See also examples 1 and 3A.

Scoring concept 2 was used for this example being a non-linear cut-off equation: Based on the [NH₄⁺]ᵤ and pH relationship and a non-linear cut-off equation between SA and non-SA.

non-SA when log([NH₄⁺]ᵤ)*(pHᵤ³)/10>17.93

or non-SA when((log([NH₄⁺]ᵤ)*(pHᵤ³)/10))−17.93>0

In FIG. 5A, [NH₄⁺]ᵤ (mM) was plotted as a function of urine pH in CKD patients and healthy controls. The inclusion criteria for the selection of CKD patients comprised a normal systemic acid/base status, i.e. no apparent metabolic acidosis. “No apparent metabolic acidosis” is defined herein as the standard HCO₃ concentration ≥22 mmol/l or total CO₂ under 22 mmol/l). In other words, these patients are not considered having apparent metabolic acidosis and are therefore CKD patients that comprise a normal systemic acid/base status, i.e. that have no apparent metabolic acidosis.

The number of observations: CKD 82 and control 25 urine samples.

Based on the identification that a large number of CKD patient points have very low urine [NH₄⁺] and very acidic urine pH, a linear cut-off line as shown above was added to FIG. 5A, as calculated based on scoring concept 2. This cut-off line was surprisingly able to fully separate the CKD patients with apparent acid retention from those CKD patients with similar urine [NH₄⁺] but at more alkaline urine i.e. a urine with higher pH. The latter group was much more alike to the urine measurements of healthy controls (black).

The non-linear cut-off line shown in FIG. 5A permitted a subdivision of CKD patients with a urine analysis indicating acid retention (left of the curve) to have SA, from a group that was apparently non-acid retaining (non-SA). Accordingly, these patients were scored to have SA or non-SA, respectively. The presented data were obtained from 24 h urine collections.

In FIG. 5B, data from 82 CKD patients are shown where kidney function loss (ΔmGFR) was plotted against the SA-scoring identified from the data in FIG. 5A. In all CKD patients (SA or Non-SA patients), the progression of kidney function loss (ΔmGFR) was followed for 1.5 years i.e. 18 months. A negative value of ΔmGFR indicates further kidney function loss.

TABLE 7
Number of patients used in FIG. 5B

| Division of CKD patients into SA score | SA | Non-SA |
|---|---|---|
| Number of patients | 51 | 31 |

In FIG. 5C, data from 82 CKD patients are shown where the estimated GFR is shown as a function of follow-up-up time in years. The development of GFR among CKD patients scored SA or non-SA is shown.

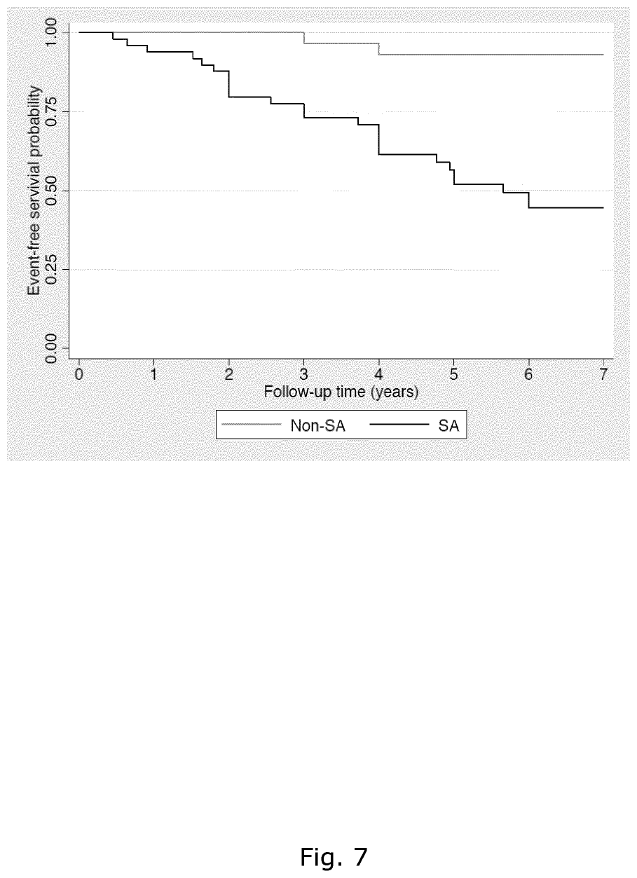

In FIG. 7, data from FIG. 5A is plotted as event-free survival probability of SA and non-SA scored CKD patients (urine NH₄⁺ vs. urinary pH) in a Kaplan-Meyer plot that displays the probability of serious clinical event-free survival (further 50% reduction in GFR, entering dialysis or kidney transplantation) of the SA and non-SA scored CKD patients measured each year up to 7 years. Patients scored as SA or non-SA based on urinary [NH₄⁺] pH relationship and the linear cut-off displayed in FIG. 5A.

### Results

FIG. 5A-C and FIG. 7:

FIG. 5A) Relationship between urinary NH₄⁺ concentration and urinary pH with the suggested non-linear cut-off equation between the score of SA or non-SA.

FIG. 5B) Development of GFR among CKD patients scored SA or non-SA. Please note that CKD patients that scored SA display a significant reduction in measured GFR during the following 18 months i.e. progression of kidney function loss (ΔmGFR). A negative value of ΔmGFR indicates further kidney function loss.

FIG. 5C) Development of GFR among CKD patients scored SA or non-SA. Please note that CKD patients that scored SA display a significant reduction in estimated GFR during the following 7 years.

FIG. 7: Kaplan-Meyer blot displaying the probability of serious clinical event-free survival (further 50% reduction in GFR, entering dialysis or kidney transplantation) of the SA and non-SA scored CKD patients. Patients scored as SA or non-SA based on urinary [NH₄⁺] pH relationship and the cut-off equation displayed in FIG. 5A. The results are summarized in Table 8.

Table 8: Hazard ratios calculated by a cox proportional hazards model based on cut-off equation displayed in 5A (Kaplan-Meyer FIG. 7). Hazard ratio adjusted for: Age (years), sex, BMI (kg/m²), baseline eGFR (mL/min/1.73 m²), urine albumin creatinine ratio (mg/g), tCO₂ (mM), and systolic blood pressure (mmHg). Please note that patients scored SA have a 10.5 times higher risk to meet a serious clinical event than non-SA scored patients. Even after adjustment for key competing clinical risk-factors, SA scored patients have a 5.3 timer high risk of experiencing a serious clinical event.

Hazard ratios (95% CI)

| | AB score (SA yes/no) | AB score (per unit) |
|---|---|---|
| Unadjusted | 10.5 (2.5 to 44) | 0.86 (0.8 to 0.92) |
| Adjusted | 5.3 (1.2 to 24.2) | 0.87 (0.79 to 0.97) |

### Conclusion

FIG. 5A surprisingly demonstrates that it is possible to divide CKD patients to groups having SA or non-SA, respectively by way of the suggested non-linear cut-off equation line between the score of SA or non-SA when using the relationship between the urinary NH₄⁺ and urinary pH.

FIG. 5B surprisingly demonstrates that the patients with SA showed accelerated loss of kidney function within a period of 18 M compared to the non-SA group, whereas the non-SA group showed stable kidney function within a period of 18M. FIG. 5C surprisingly demonstrates that patients scored SA display a significant reduction in estimated GFR than non-SA scored patients when followed for 7 years.

FIG. 7 surprisingly demonstrates that patients that scored SA has a 10.5 times higher risk of meeting a serious clinical event than non-SA scored patients when followed for 7 years. Even after adjustment for key competing clinical risk-factors, SA scored patients has a 5.3 timer high risk of experiencing a serious clinical event.

Thus, the inventors have surprisingly identified that the relationship between urinary NH₄⁺ and urinary pH in CKD-patients can be used to identify CKD-patients, by use of the scoring concept 2, that are at high risk of developing kidney function loss (ΔmGFR) within e.g. 18 months and/or display a significant reduction in estimated GFR when followed for 7 years or meeting a serious clinical event when followed for 7 years. By early identification of such risks in said patient group, interventions to prevent kidney function loss can be initiated to avoid said risks.

### Example 3D—Dynamic Response: Dietary Intervention on the Relationship Between Urinary NH₄⁺ Concentration and Urinary pH in CKD Patients—Scoring Concept 2

### Aim

To evaluate the relevance of the SA/non-SA score for CKD patients when subjected to a dietary intervention that is known to reduce the systemic acid load and thus reduce the risk of acid overload in CKD patients.

### Materials and Methods

See also example 1 and 3C.

Scoring concept 2 was used for this example being a non-linear cut-off:

Based on the [NH₄⁺]ᵤ and pH relationship and a non-linear cut-off equation between SA and non-SA.

non-SA when log([NH₄⁺]ᵤ)*(pHᵤ³)/10>17.93

or non-SA when ((log([NH₄⁺]ᵤ)*(pHᵤ³)/10))−17.93>0

### Results

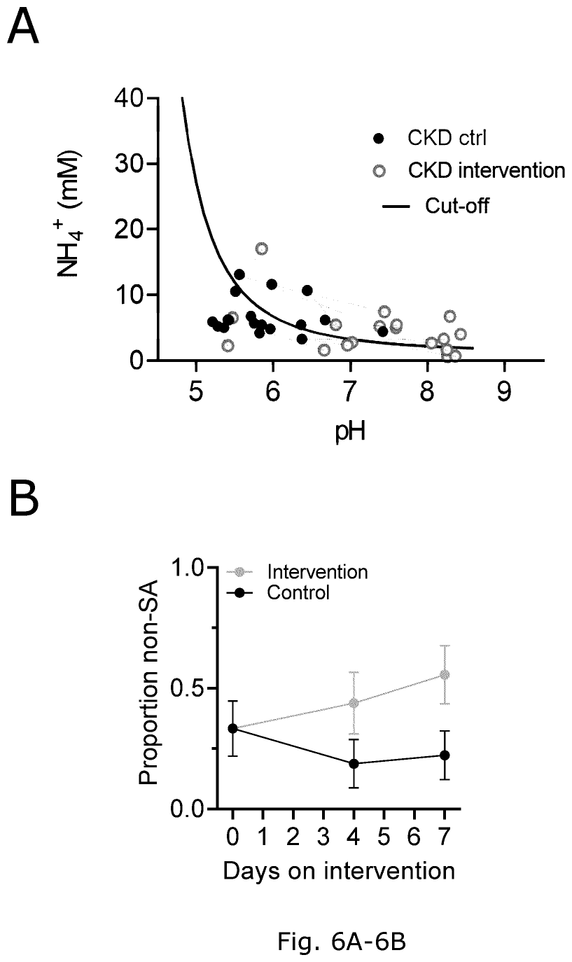

FIG. 6A-6B:

FIG. 6: A) Relationship between urinary NH₄⁺ concentration and urinary pH with the suggested non-linear cut-off equation between the score of SA or non-SA as defined in FIG. 5A. Black dots are baseline and grey dots after 1-week dietary intervention (NNRD) where two measurements from each patient are linked with thin lines. The black dots represent CDK ctrl (control); i.e. CKD levels before dietary intervention (day zero) and the grey dots represents CKD intervention i.e. CKD levels after dietary intervention (day seven). B) Proportions of CKD patients scored non-SA during a one-week acid-reducing dietary intervention.

It is surprisingly shown that the dietary intervention with the NNRD after just seven days shifts a significant number of CKD patients to the right side of the cut-off line. This means that the results show that CKD patients having SA, become non-SA after only seven days on the NNRD. These results were quantified and are shown in FIG. 6B.

FIG. 6B: The fraction of CKD patients that scored non-SA increased highly surprisingly from 33% to 56% after a week on the NNRD.

### Conclusion

Thus, surprisingly, the urine analysis appears a very sensitive measure to monitor a dynamic effect of a short term acid-reducing intervention such as the NNRD as compared to the changes in blood acid/base measures (FIGS. 6A+6B). Surprisingly, the fraction of CKD patients that scored non-SA, based on a non-linear cut-off equation (scoring concept 2), increased from about 33% to 56% after a week of dietary intervention.

### Example 4 Explores the [NH₄⁺]ᵤ and [TA] Relationship—Scoring Concept 3

### Aim

To evaluate the relevance of the relationship between the urinary NH₄⁺ concentration and the titratable acid (TA) concentration with the suggested non-linear cut-off equation between the score of SA or non-SA.

### Materials and Methods

See also example 1.

Scoring concept 3 was used for this example:

Based on the [NH₄⁺]ᵤ and [TA] relationship and a non-linear cut-off equation between SA and non-SA.

non-SA when log [NH₄⁺]ᵤ*log(([TA]ᵤ*−1)+28)<0.926

or non-SA when (log [NH₄⁺]ᵤ*log(([TA]ᵤ*−1)+28)−0.926<0

In this example, 28 is added to avoid negative TA values, to allow log transformation. It will of course depend on what the wanted absolute lower range of TA is how much that needs to be added (if a scoring system comprising TA values lower than 28 is wanted, more than 28 needs to be added).

TABLE 9
Number of patients used in FIG. 8B

| Division of CKD patients into SA score | SA | Non-SA |
|---|---|---|
| Number of patients | 26 | 56 |

### Results

FIG. 8A-8B:

FIG. 8A: Relationship between urinary NH₄⁺ concentration and urinary TA with the suggested non-linear cut-off equation between the score of SA or non-SA. The non-SA are shown on the left of the curve thus having less TA than CKD patients that scored SA.

FIG. 8B: Development of GFR among CKD patients scored SA or non-SA. Please note that CKD patients that scored SA display a significant reduction in estimated GFR during the following 6 years.

FIG. 9: Kaplan-Meyer blot displaying the probability of serious clinical event-free survival (further 50% reduction in GFR, entering dialysis or kidney transplantation) of the SA and non-SA scored CKD patients. Patients scored as SA or non-SA based on urinary [NH₄⁺] TA relationship and the cut-off equation displayed in FIG. 8A. The results are summarized in Table 10.

Table 10: Hazard ratios calculated by a cox proportional hazards model based on cut-off equation displayed in FIG. 8A (Kaplan-Meyer FIG. 9). Hazard ratio adjusted for: Age (years), sex, BMI (kg/m²), baseline eGFR (mL/min/1.73 m²), urine albumin creatinine ratio (mg/g), tCO2 (mM), and systolic blood pressure (mmHg). Please note that patients scored SA has a 2.3 times higher risk to meet a serious clinical event than non-SA scored patients. Even after adjustment for key competing clinical risk-factors, SA scored patients has a 2.5 timer high risk of experiencing a serious clinical event.

Hazard ratios (95% CI)

| | SA score (SA yes/no) | SA score (per unit) |
|---|---|---|
| Unadjusted | 2.3 (1.1 to 4.9) | 0.47 (0.2 to 1.1) |
| Adjusted | 2.5 (1.1 to 5.7) | 0.29 (0.09 to 0.94) |

### Conclusion

FIG. 8A surprisingly demonstrate that it is possible to divide CKD patients to groups having SA or non-SA, respectively by way of the suggested non-linear cut-off line calculated by use of the scoring concept 3 when using the relationship between the urinary NH₄⁺ concentration and the titratable acid (TA) concentration.

FIG. 8B surprisingly demonstrates that patients scored SA display a significant reduction in estimated GFR than non-SA scored patients when followed for 6 years.

FIG. 9 surprisingly demonstrates that patients scored SA have a 2.3 times higher risk of meeting a serious clinical event than non-SA scored patients when followed for 7 years. Even after adjustment for key competing clinical risk-factors, SA scored patients have a 2.5 timer high risk of experiencing a serious clinical event.

Thus, the inventors have surprisingly identified that the relationship between urinary NH₄⁺ and urinary TA in CKD-patients based on scoring concept 3 can be used to identify CKD-patients that display a significant reduction in estimated GFR when followed for 6 years or meeting a serious clinical event when followed for 7 years. By early identification of such risks in said patient group, interventions to prevent kidney function loss can be initiated to avoid said risks.

### Example 5 Explores the Relationship Between the Urinary NH₄⁺ Excretion and the Urinary Total Base Excretion Rate—Scoring Concept 4

### Example 5A

### Aim

To evaluate the relevance of the relationship between the NH₄⁺ excretion and the total base (TB) excretion with the suggested linear cut-off line between the score of SA or non-SA.

### Materials and Methods

See also example 1.

To further assess the possible value of the suggested SA score, the inventors analyzed the function of the NH₄⁺ urinary excretion versus the urine total base excretion in CKD patients and healthy controls. The presented data were obtained from 24 h urine collections.

Scoring concept 4 was used for this example:

Based on the relationship between NH₄⁺ and total base excretion.

non-SA when NH₄⁺excretion≥−1*TB excretion+0

or non-SA when TB excretion is ≥0

TABLE 11
Number of patients used in FIG. 10B

| Division of CKD patients into SA score | SA | Non-SA |
|---|---|---|
| Number of patients | 29 | 52 |

### Results

FIG. 10A: Relationship between urinary NH₄⁺ excretion and urinary total base excretion rate (TB=([BB]+ [HCO₃⁻])*urine volume) with the suggested cut-off line between the score of CKD patients into SA or non-SA.

FIG. 10B: Development of GFR among CKD patients scored SA or non-SA. Please note that CKD patients scored SA display a significant reduction in GFR during the following 18 months.

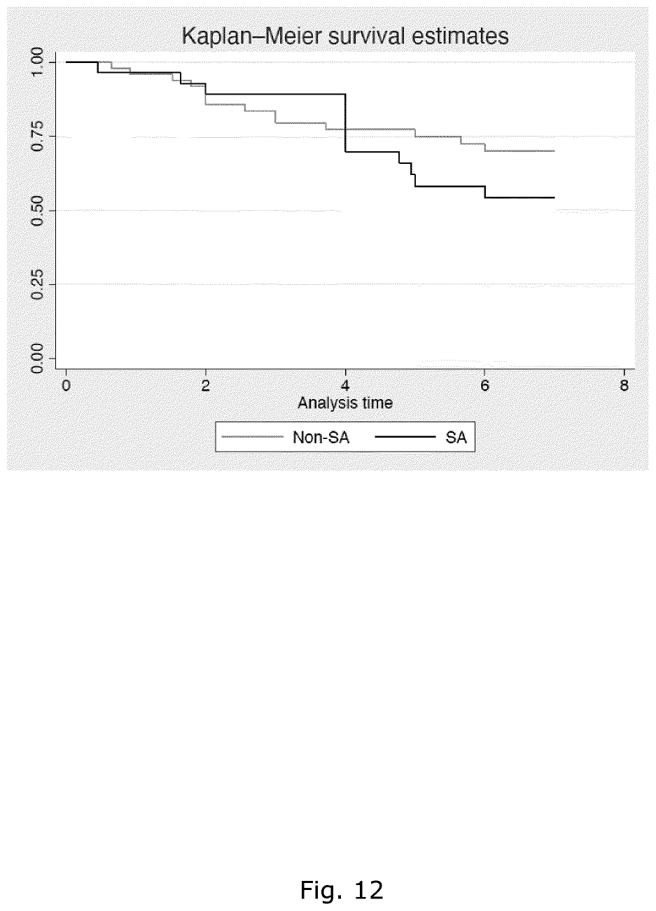

FIG. 12: Kaplan-Meyer blot displaying the probability of serious clinical event-free survival (further 50% reduction in GFR, entering dialysis or kidney transplantation) of the SA and non-SA scored CKD patients. Patients scored as SA or non-SA based on the cut-off equation displayed in FIG. 10A. The results are summarized shown in Table 12.

Table 12: Hazard ratios calculated by a cox proportional hazards model based on cut-off equation displayed in FIG. 10A (Kaplan-Meyer FIG. 12). Hazard ratio adjusted for: Age (years), sex, BMI (kg/m²), baseline eGFR (mL/min/1.73 m²), urine albumin creatinine ratio (mg/g), tCO2 (mM), and systolic blood pressure (mmHg). Please note that patients scored SA has a 1.48 times higher risk to meet a serious clinical event than non-SA scored patients. Even after adjustment for key competing clinical risk-factors, SA scored patients have a 1.31 times high risk of experiencing a serious clinical event.

Hazard ratios (95% CI)

| | SA score (SA yes/no) | SA score (per unit) |
|---|---|---|
| Unadjusted | 1.48 (0.68 to 3.21) | 0.97 (0.95 to 1.00) |
| Adjusted | 1.31 (0.54 to 3.22) | 0.98 (0.95 to 1.01) |

### Conclusion

FIG. 10A surprisingly demonstrate that it is possible to divide CKD patients to groups having SA or non-SA, respectively by way of the suggested linear cut-off line between the score of SA or non-SA when using of the relationship between the NH₄⁺ excretion and the total base (TB) excretion.

FIG. 10B surprisingly demonstrate that the patients with SA showed accelerated loss of kidney function within a period of 18 M compared to the non-SA group, whereas the non-SA group showed stable kidney function within a period of 18M.

FIG. 12 surprisingly demonstrate that patients scored SA has a 1.48 times higher risk of meeting a serious clinical event than non-SA scored patients when followed for 7 years. Even after adjustment for key competing clinical risk-factors, SA scored patients has a 1.31 times high risk of experiencing a serious clinical event.

Thus, the inventors have surprisingly identified that the relationship between urinary NH₄⁺ excretion and urinary total base excretion rate in CKD-patients based on scoring concept 4 can be used to identify CKD-patients that are at high risk of developing kidney function loss (ΔmGFR) within e.g. 18 months and/or meeting a serious clinical event when followed for 7 years.

By early identification of such risks, interventions to prevent kidney function loss can be initiated to avoid said risks in said patient group.

### Example 5B—Dynamic Response: Dietary Intervention on the Relationship Between the NH₄⁺ Excretion and the Total Base (TB) Excretion in CKD Patients—Scoring Concept 4

### Aim

To evaluate the relevance of the SA/non-SA score for CKD patients when subjected to a dietary intervention that is known to reduce the systemic acid load and thus reduce the risk of acid overload in CKD patients.

### Materials and Methods

See also example 1.

Scoring concept 4 was used for this example:

Based on the relationship between NH₄⁺ and total base excretion.

non-SA when NH₄⁺excretion≥−1*TB excretion+0

or non-SA when TB excretion is ≥0

The inventors analyzed CKD patients before and after a short-term acid-reducing dietary intervention (New Nordic Renal Diet, NNRD) i.e. 1 week (seven days) intervention. The NNRD is known to reduce the systemic acid load and thus reduce the risk of acid overload in CKD patients. Thus, the urinary NH₄⁺ excretion and urinary TB excretion were measured in all CKD patients before and after the short-term dietary intervention. In order to quantify the observations, the inventors added a linear “cut-off” line [NH₄⁺ excretion ≥−1*TB excretion+0], as shown in FIG. 11A. Black lines connecting the CKD ctrl to CKD intervention is not shown in FIG. 11A (to improve readability of figure).

### Results

FIG. 11A) Relationship between urinary NH₄⁺ excretion and urinary TB excretion with the suggested linear cut-off line between the score of SA or non-SA. Black dots are baseline and grey dots after 1-week dietary intervention.

FIG. 11B) Proportions of CKD patients scored non-SA during a one-week acid-reducing dietary intervention. Please note that the proportion of non-SA-scored patients increased already at day 4. The fraction of CKD patients that scored non-SA, based on the linear cut-off line, increased from about 11% to 61% after a week of dietary intervention.

### Conclusion

Surprisingly, the urine analysis based on scoring concept 4 appears a very sensitive measure to monitor a dynamic effect of a short-term acid-reducing intervention such as the NNRD as compared to the changes in blood acid/base measures (FIGS. 11A+11B). Surprisingly, the fraction of CKD patients that scored non-SA, based on a linear cut-off line, increased from about 11% to 61% after a week of dietary intervention.

### Example 6 Explores the Relationship Between the Relative (Normalized to Creatinine) NH₄⁺ and Base Buffer/Bicarbonate Excretion

24 h urine collection is cumbersome and difficult to adapt to the workflow in clinical practice. Thus for practical usability, a SA score concept that is applicable on simple spot urine samples would be preferable.

The inventors did not collect spot urines from any CKD patient cohort, nevertheless the following data in examples 6A and 6B surprisingly demonstrate that the SA scoring system is applicable to spot urines. Spot urines vary in volume and thus concentration/dilution depending on the hydration status of the subject.

To overcome these variations spot urine samples can be normalized to the amount of creatinine in the spot urine samples. Accordingly, normalization to the amount of creatinine in the spot urine samples were performed in these examples. Creatinine is an endogenously produced substance that is excreted by the kidneys at a constant rate. 24H urine collections can be used as spot urine samples. Normalization can be done by expressing the urine acid/base biomarkers over the urine creatinine concentration. Resultantly, in these 24 h derived spot urines, it is possible to make a functional separate between CKD patients and healthy controls on the level of urinary acid/base biomarkers.

### Example 6A—the Relative (Normalized to Creatinine) NH₄⁺ and Base Buffer-Scoring Concept 5

### Aim

To evaluate the relevance of the relationship between the relative (normalized to creatinine) NH₄⁺ and relative (normalized to creatinine) base buffer excretion with the suggested linear cut-off line between the score of SA or non-SA.

### Materials and Methods

See also example 1.

The scoring concept 5 was used for this example:

Based on the relationship between the relative (normalized to creatinine) NH₄⁺ excretion and relative (normalized to creatinine) base buffer (BB) excretion:

non-SA when [NH₄⁺]ᵤ/[creatinine]ᵤ≥−2.5*[BB]ᵤ/[Creatinine]ᵤ+0

or non-SA when [BB]ᵤ/[Creatinine]ᵤ is ≥0

TABLE 13
Number of patients used in FIG. 14

| Division of CKD patients into SA score | SA | Non-SA |
|---|---|---|
| Number of patients | 54 | 26 |

### Results

FIG. 13A: Relationship between urinary NH₄⁺ creatinine ratio and BB creatinine ratio with the suggested cut-off line between the score of SA or non-SA.

FIG. 14: Kaplan-Meyer blot displaying the probability of serious clinical event-free survival (further 50% reduction in GFR, entering dialysis or kidney transplantation) of the SA and non-SA scored CKD patients. Patients scored as SA or non-SA based on the cut-off equation displayed in FIG. 13A. The results are summarized in Table 14.

Table 14: Hazard ratios calculated by a cox proportional hazards model based on cut-off equation displayed in FIG. 13A (Kaplan-Meyer FIG. 14). Hazard ratio adjusted for: Age (years), sex, BMI (kg/m²), baseline eGFR (mL/min/1.73 m²), urine albumin creatinine ratio (mg/g), tCO₂ (mM), and systolic blood pressure (mmHg). Please note that patients scored SA has a 2.16 times higher risk to meet a serious clinical event than non-SA scored patients. Even after adjustment for key competing clinical risk-factors, SA scored patients has a 1.02 times high risk of experiencing a serious clinical event.

Hazard ratios (95% CI)

| | SA score (SA yes/no) | SA score (per unit) |
|---|---|---|
| Unadjusted | 2.16 (0.82 to 5.75) | 0.89 (0.77 to 1.02) |
| Adjusted | 1.02 (0.34 to 3.02) | 0.97 (0.83 to 1.14) |

### Conclusion

FIG. 13A surprisingly demonstrate that it is possible to divide CKD patients to groups having SA or non-SA, respectively by way of the suggested linear cut-off line between the score of SA or non-SA based on the relationship between the relative (normalized to creatinine) NH₄⁺ and relative (normalized to creatinine) base buffer excretion.

FIG. 14 surprisingly demonstrate that patients scored SA has a 2.16 times higher risk of meeting a serious clinical event than non-SA scored patients when followed for 7 years. Even after adjustment for key competing clinical risk-factors, SA scored patients has a 1.02 times high risk of experiencing a serious clinical event.

Thus, the inventors have surprisingly identified that the relationship between urinary NH₄⁺ creatinine ratio and BB creatinine ratio in CKD-patients can be used to identify CKD-patients, by use of the scoring concept 5, that are at high risk of meeting a serious clinical event when followed for 7 years.

By early identification of such risks, interventions to prevent kidney function loss can be initiated to avoid said risks, which has been shown here to be possible using spot-urine samples.

### Example 6B—the Relative (Normalized to Creatinine) NH₄⁺ and Bicarbonate Excretion—Scoring Concept 6

### Aim

Further, to evaluate the relevance of the relationship between the relative (normalized to creatinine) NH₄⁺ and the relative (normalized to creatinine) bicarbonate excretion with the suggested linear cut-off line between the score of SA or non-SA.

### Materials and Methods

See also example 1.

The scoring concept 6 was used for this example:

Based on the relationship between the relative (normalized to creatinine) NH₄⁺ excretion and relative HCO₃-excretion:

non-SA when [NH₄⁺]ᵤ/[creatinine]ᵤ≥−150*[HCO₃⁻]ᵤ/[Creatinine]ᵤ+60

or non-SA when [HCO₃⁻]ᵤ/[Creatinine]ᵤ is ≥0.

TABLE 15
Number of patients used in FIG. 15

| Division of CKD patients into SA score | SA | Non-SA |
|---|---|---|
| Number of patients | 70 | 10 |

### Results

FIG. 13B: Relationship between urinary NH₄⁺ creatinine ratio and urinary HCO₃⁻ creatinine ratio with the suggested linear cut-off line between the score of SA or non-SA.

FIG. 15: Kaplan-Meyer blot displaying the probability of serious clinical event-free survival (further 50% reduction in GFR, entering dialysis or kidney transplantation) of the SA and non-SA scored CKD patients. Patients scored as SA or non-SA based on the linear cut-off equation displayed in FIG. 13B. The results are summarized in Table 16.

Table 16: Hazard ratios calculated by a cox proportional hazards model based on cut-off equation displayed in FIG. 13B (Kaplan-Meyer FIG. 15). Hazard ratio adjusted for: Age (years), sex, BMI (kg/m²), baseline eGFR (mL/min/1.73 m²), urine albumin creatinine ratio (mg/g), tCO₂ (mM), and systolic blood pressure (mmHg). Please note that patients scored SA has a 1.60 times higher risk to meet a serious clinical event than non-SA scored patients. Even after adjustment for key competing clinical risk-factors, SA scored patients has a 0.72 times high risk of experiencing a serious clinical event.

Hazard ratios (95% CI)

| | SA score (SA yes/no) | SA score (per unit) |
|---|---|---|
| Unadjusted | 1.60 (0.38 to 6.79) | 0.99 (0.98 to 1.01) |
| Adjusted | 0.72 (0.14 to 3.8) | 1.00 (0.99 to 1.02) |

### Conclusion

FIG. 13B surprisingly demonstrate that it is possible to divide CKD patients to groups having SA or non-SA, respectively by way of the suggested linear cut-off line between the score of SA or non-SA.

FIG. 15 surprisingly demonstrate that patients scored SA has a 1.60 times higher risk of meeting a serious clinical event than non-SA scored patients when followed for 7 years. Even after adjustment for key competing clinical risk-factors, SA scored patients has a 0.72 times high risk of experiencing a serious clinical event.

Thus, the inventors have surprisingly identified that the relationship between urinary NH₄⁺ creatinine ratio and urinary HCO₃ creatinine ratio in CKD-patients by use of the scoring concept 6 can be used to identify CKD-patients that are at high risk of meeting a serious clinical event when followed for 7 years.

By early identification of such risks, interventions to prevent kidney function loss can be initiated to avoid said risks, which has been shown here to be possible using spot-urine samples.

### Summary Conclusions from Examples 1-6

In summary, the data presented in FIGS. 1-6 strongly suggest that acid retention and subclinical acidosis (SA) is a clinically relevant measure that can be easily detected in simple urine collections from CKD patients. SA scores correlate with disease progression and the SA score is a dynamic parameter that is influenced by a relevant dietary intervention.

Data has been presented where urinary NH₄⁺ concentration was plotted as a function of four different biomarkers of acid excretion demand. These were:
urinary pH,
urinary titratable acids (TA),
urinary total base (TB),
urinary base buffers (BB),
urinary HCO₃⁻.

The inventors suggested that acid accumulation in CKD can be assessed as ratio analyses of urinary NH₄⁺ and any measure of the demand for acid excretion.

All presented SA scoring concepts are based on the relationship between two urinary biomarkers.

A variety of scoring concepts were tested in order to support the broad concept of being able to identify acid retention and subclinical acidosis (SA) as a clinically relevant measure that can be easily detected in simple urine collections from CKD patients. The Kaplan-Meier survival estimate plots strongly supports this clinically relevant scoring system of CKD patients in SA or non-SA groups.

### Example 7—Further Evaluation of Scoring Concept 2, Variant “AB_Score”

### Aim

To establish the relevance of the relationship between urine NH₄⁺ and urine pH in CKD patients and healthy controls.

Further, to demonstrate the variability and thus suitability of the used AB_score to distinguish CKD patients into SA or non-SA groups in the study group NCT03052582 (RENVAS).

Further, to validate the AB_score using the study group NCT (PUMA).

Lastly, to assess intra-individual variability of the AB_score during a 6-month period with repeated 24 h urine collections using the study (NNRD).

### Materials and Methods

### Samples:

CKD patients from three clinical studies (RENVAS, PUMA, and NNRD33) were included. The RENVAS study (clinical trials registry number: NCT01380717) is presented in “Example 1—Study design, participation and methods used for data collection and calculations” to which is referred.

All three studies included patients with CKD stage 3 and 4 who had 24 h urine collections available for acid/base analysis from the time of inclusion.

The RENVAS study cohort was used as the development cohort to establish a urine acid/base score (AB_score) and evaluate its ability to predict CKD progression as shown in Examples 1-6 herein.

The PUMA study cohort examined mechanisms responsible for albuminuria. The PUMA study cohort was included as validation cohort to evaluate the AB_score developed in the RENVAS cohort and the specific cut-off to indicate subclinical acidosis.

NNRD (clinical trials registry number: NCT04579315) studied the effect of a 6-month dietary intervention (New Nordic Renal Diet) on phosphate homeostasis. The control group of the study was used to assess intra-individual variability of the AB_score during a 6-month period with repeated 24 h urine collections.

The study population with urine acid-base parameters measured consisted of 73 (PUMA), and 59 (NNRD) participants with CKD.

Baseline characteristics of the PUMA and NNRD groups are shown in Table 17.

TABLE 17
Baseline characteristics of the development cohort
(RENVAS), validation cohort (PUMA), the variation
cohort (NNRD), and control cohort (RENVAS).

| | | | NNRD | |
|---|---|---|---|---|
| | PUMA | | CKD | |
| | CKD (n = 73) | | (n = 59) | |
| Age, y (IQR) | 71.4 | (65.3-74.9) | 54 | (46-66) |

| | | |
|---|---|---|
| Female, % | 36.9% | 47.5% |

| | | | | |
|---|---|---|---|---|
| BMI, kg/m² (SD) | 29.2 | (5.6) | 26 | (4.3) |
| eGFR, | 40.2 | (15.1) | 36.2 | (9.9) |
| mL/min/1.73 m² (SD) | | | | |

| | | |
|---|---|---|
| mGFR, | NA | NA |

| mL/min/1.73 m² (SD) | | | | |
|---|---|---|---|---|
| Venous tCO₂, mM | 24.9 | (3.6) | 22.5 | (3) |
| (SD) | | | | |
| Urine ACR, mg/g | 48 | (10-405) | NA | |
| (IQR) | | | | |

Urine ACR (UACR) means Urine Albumin (mg/dL)/Urine Creatinine (g/dL)=UACR in mg/g≈Albumin excretion in mg/day.

UACR is a ratio between two measured substances. Unlike a dipstick test for albumin, UACR is unaffected by variation in urine concentration.

Albuminuria is present when UACR is greater than 30 mg/g and is a marker for CKD.

### Data Collections:

For PUMA, the inventors collected data on GFR, time of initiation of chronic dialysis or renal transplantation, and death from inclusion until the last follow-up. Information on GFR was collected yearly (+/−3 months). Participants in the PUMA study were followed for up to 10 years.

Measurements of Urine pH and NH₄⁺:

We refer to information in Example 1.

### Urine Acid/Base Score (AB Score):

Based on measured urine pH and ammonium, a urine AB_score was calculated. The following equation provides the strongest association between the urine AB_score and both urinary [NH₄⁺] and urinary pH:

$$AB\_ score = \frac{log [ {NH}_{4}^{+} ] \cdot {pH}^{3}}{10}$$

### Results

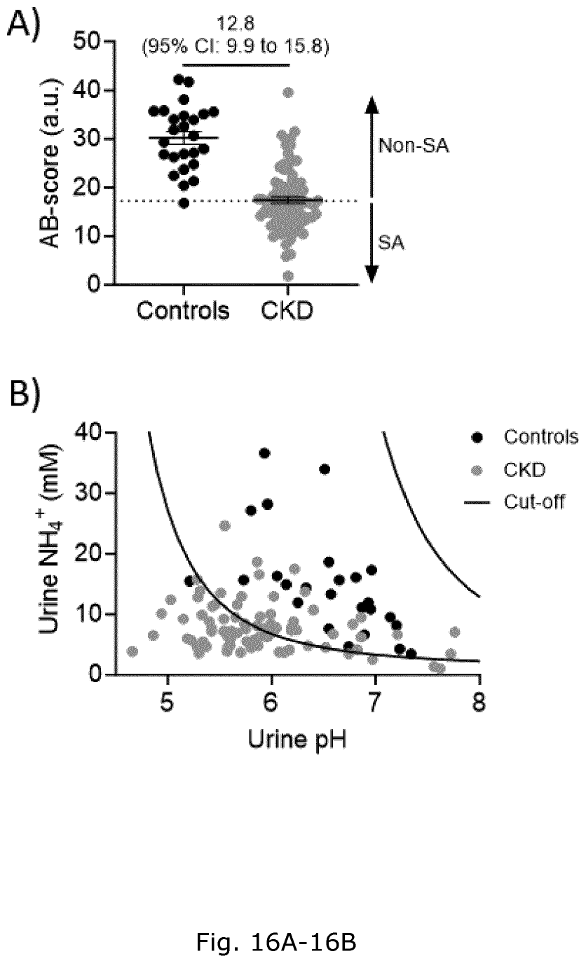

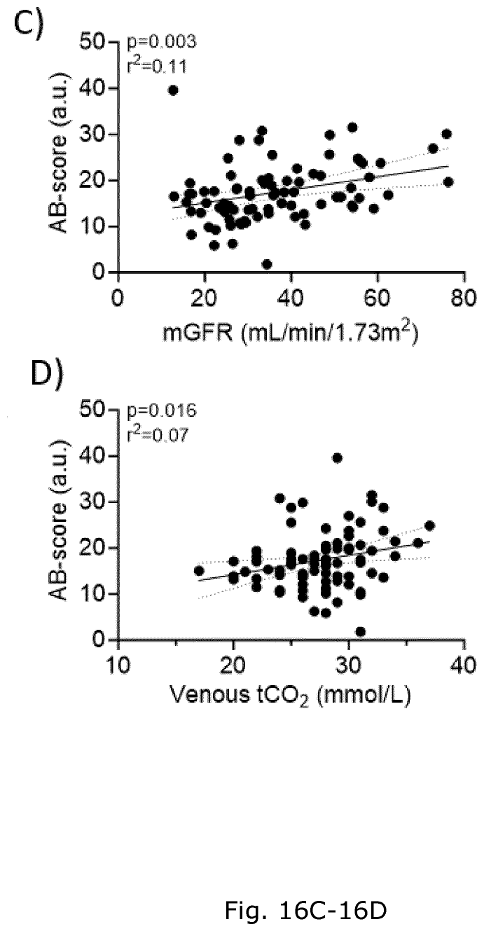

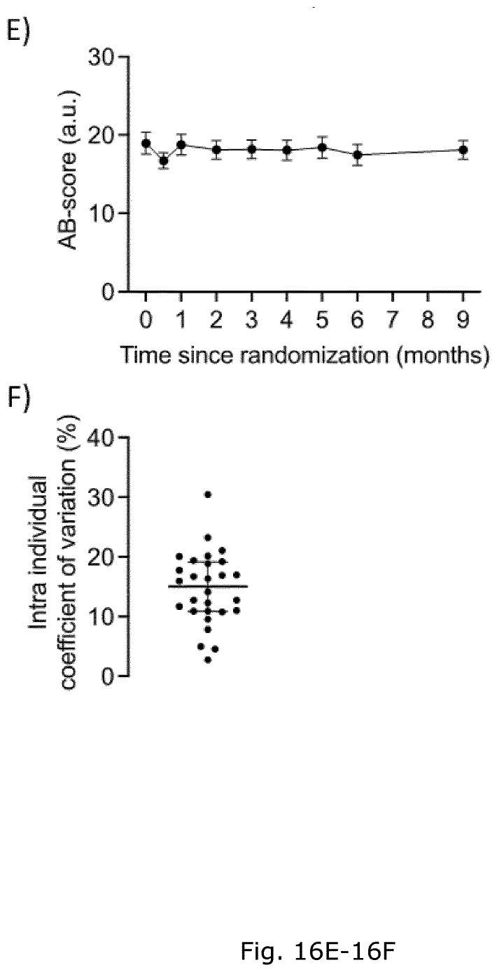

AB_score was higher in control participants (12.8 a.u., 95% CI: 9.9-15.8, FIG. 16A). Using the lower 2.5th percentile of control participants as a cut-off to define SA (FIG. 16A-B), 62% of CKD participants were considered to have SA in the development cohort (RENVAS). In this cohort, the AB_score associated only poorly with mGFR (r2=0.11) and plasma tCO₂ (r2=0.07) (FIG. 16C-D). Only 6.2% had metabolic acidosis, as reflected by plasma tCO2<22 mmol/L, all of which were classified as SA and were in the lowest AB_score tertile. Baseline characteristics in the development and validation cohort (PUMA) stratified for AB_score tertile.

In the control group of the NNRD cohort, 24 h urine collections were collected at baseline and at eight consecutive visits during a nine-month period. During this period, the AB_score was relatively stable with a mean intra-individual coefficient of variation of 14.6% (95% CI: 12.3-17, FIG. 16E-F).

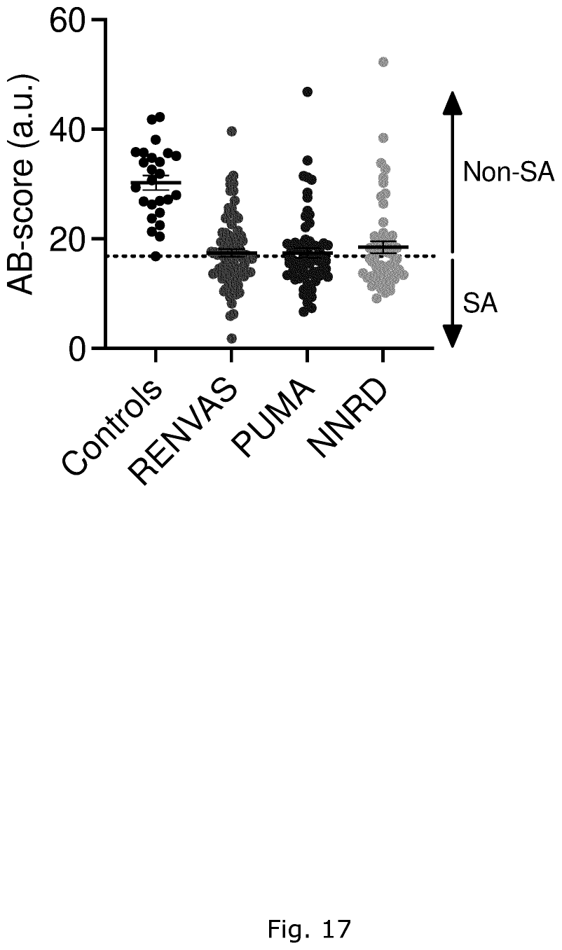

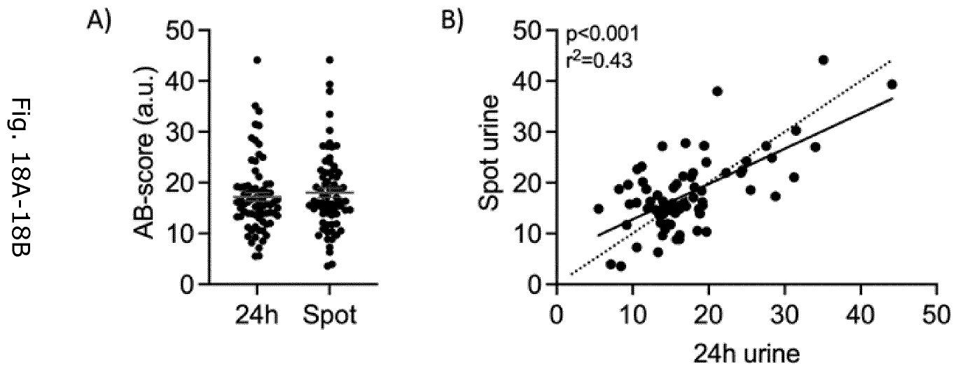

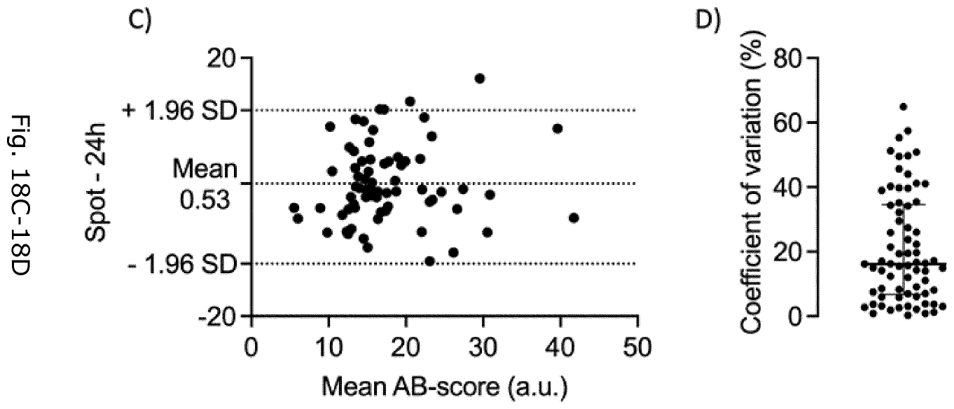

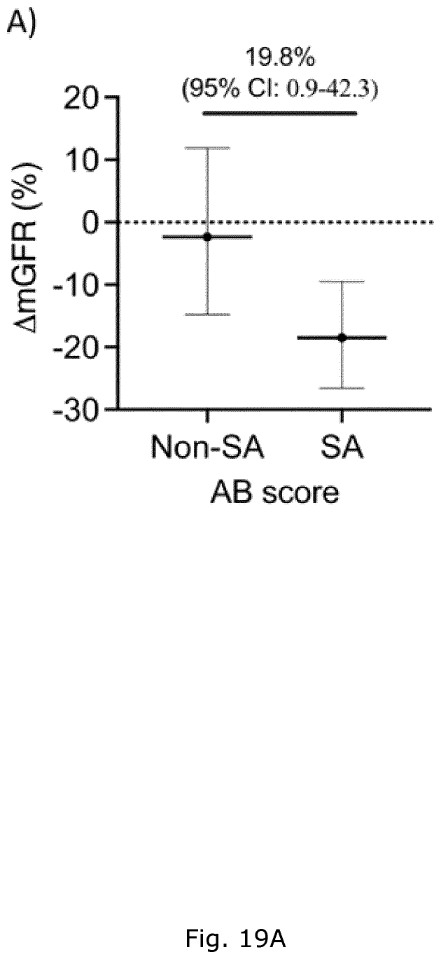

Interestingly, no apparent differences were found between the AB_score of urine samples stored at −20° C. for up to 10 years (RENVAS and PUMA) and samples stored at −20° C. for a much shorter time, i.e., months (NNRD) (FIG. 17). In the PUMA cohort, both spot and 24 h urine collections were available. No systematic bias was found between the AB_score in spot and 24 h collections (FIG. 18A-C).

However, the median coefficient of variation was 16.2% and 25% had a deviation >34.5% from between spot and 24 h urine (FIG. 18D).

### CKD Progression in the RENVAS Development Cohort

In the RENVAS cohort, the unadjusted hazard ratio (HR) for progression of CKD defined as a ≥50% decrease in eGFR, initiation of chronic dialysis or renal transplantation, was 10.5 (95% CI: 2.5-44) for patients classified as SA compared to non-SA (Table 18, see below).

Consistent with this, the unadjusted HR for a CKD progression was 0.36 (95% CI: 0.23 to 0.57) per SD increase in AB_score (Table 18). After adjustment for age, sex, BMI, systolic blood pressure, urine ACR, baseline eGFR, and tCO₂, SA status and a lower AB_score remained strongly associated with a higher risk for CKD progression.

The adjusted HRs were 5.3 (95% CI: 1.2-24.2) for patients classified at SA compared to non-SA and 0.41 (95% CI: 0.21-0.79) per SD higher AB_score (Table 18). Exclusion of acidotic patients or inclusion of acidosis (yes vs. no) as a covariable instead of tCO₂ did not impact the findings (data not shown).

We also refer to table 8, demonstrating the same data for the RENVAS study albeit with minor differences due to variations in statistical methods and approximations.

TABLE 18
Hazard ratios from a cox proportional hazard
model for CKD progression based on AB_score
in the development (RENVAS) and validation (PUMA) cohort.
The first column contains hazard ratios based on
the 2.5th percentile of control participants as
a cut-off signifying subclinical acidosis
(SA). The second column shows hazard ratio
reduction per SD higher AB score.

| | Hazard ratios (95% CI) | | | |
|---|---|---|---|---|
| | AB_score | | AB_score | |
| | (SA yes | | (per SD | |
| Cohort | vs. no) | P | higher) | P |
| RENVAS | | | | |
| (n = 82) | | | | |
| Unadjusted | 10.5 (2.5 to 44) | 0.001 | 0.36 (0.23 to 0.57) | <0.001 |
| Adjusted^{a} | 5.4 (1.2 to 24.4) | 0.030 | 0.41 (0.21 to 0.79) | 0.009 |
| PUMA | | | | |
| (n = 73) | | | | |
| Unadjusted | 3.4 (1.1 to 10) | 0.029 | 0.59 (0.35 to 1.01) | 0.054 |
| Adjusted^{a} | 8.6 (2.3 to 32) | 0.001 | 0.43 (0.23 to 0.78) | 0.006 |

^{a}Adjusted for age (years), sex, BMI (kg/m²), baseline eGFR (mL/min/1.73 m²), UACR (mg/g), systolic blood pressure (mmHg), and tCO₂ (mM)

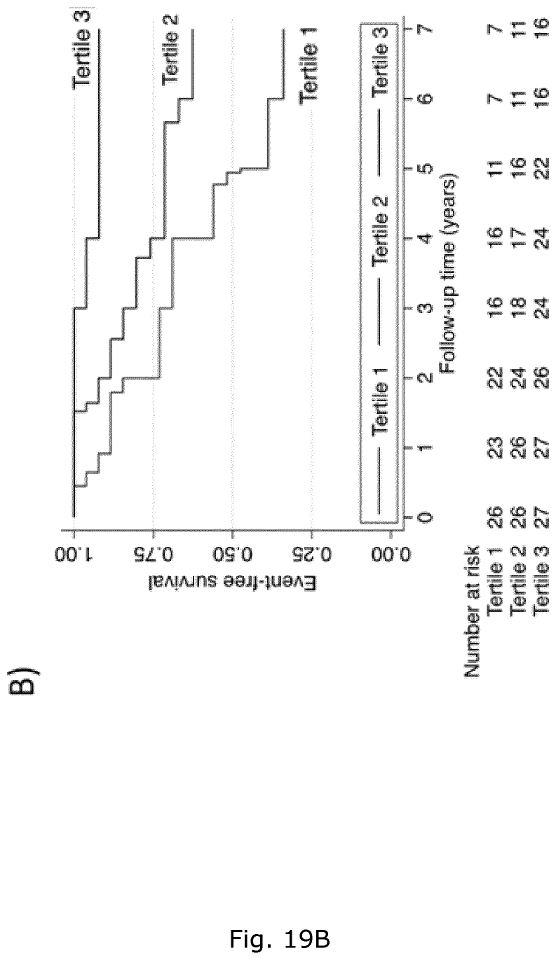

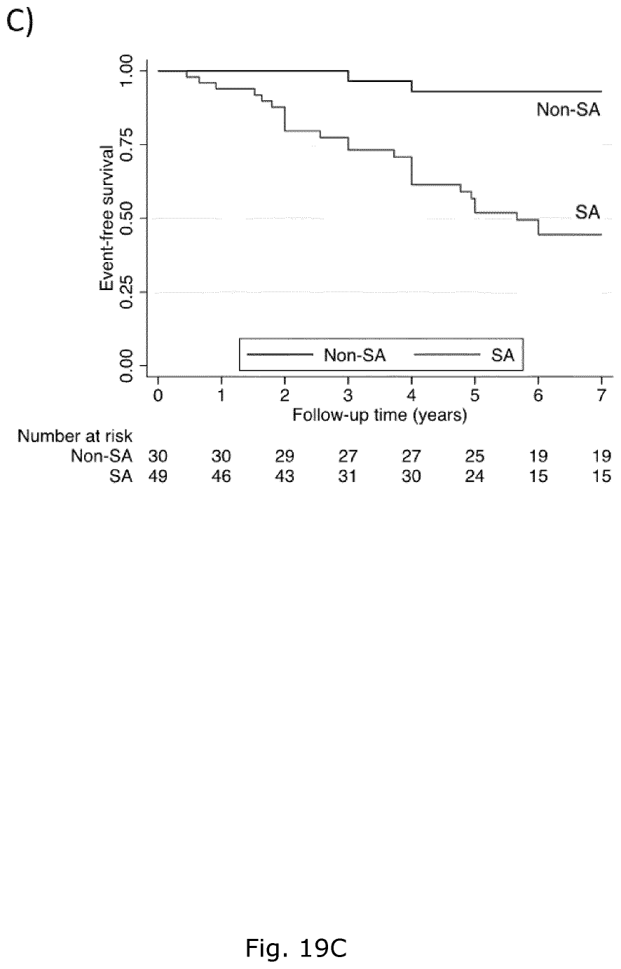

The event-free survival stratified for AB_score tertile and SA status is illustrated by Kaplan-Meier plots (FIG. 19B and FIG. 7).

### CKD Progression in the PUMA Validation Cohort

A higher AB_score and non-SA status were associated with a lower risk for CKD progression similarly defined as a ≥50% decrease of eGFR, initiation of chronic dialysis or renal transplantation.

In adjusted cox proportional hazards models, the HRs were 8.6 (95% CI: 2.3-32) for patients classified as SA compared to non-SA and 0.43 (95% CI: 0.23-0.78) per SD higher AB_score (Table 18).

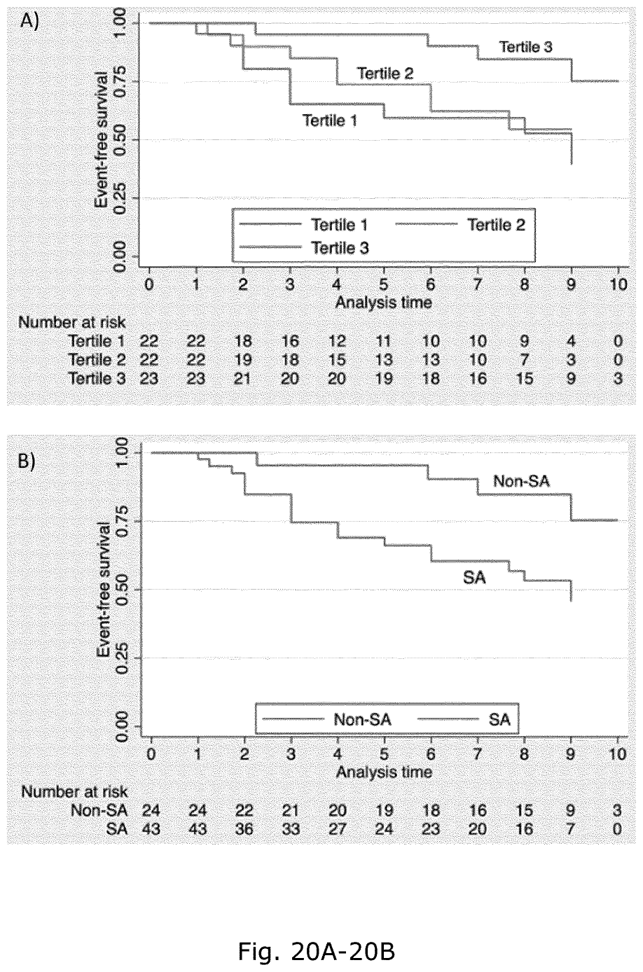

The event-free survival stratified for AB_score tertile, and SA status is illustrated by Kaplan-Meier plots in FIG. 20A-B.

### Analysis of the Pooled RENVAS and PUMA Cohort

An analysis pooling the two cohorts yielded similar adjusted HRs, but with improved confidence intervals, i.e. at 5.6 (95% CI: 2.2 to 14.1) for patients classified as SA compared to non-SA and at 0.46 (95% CI: 0.30 to 0.70) per SD higher AB_score (see Table 19).

TABLE 19
Hazard ratios for CKD progression (reaching the composite outcome)
based on AB_score in a pooled analysis of RENVAS and PUMA. The first column
contains hazard ratios based on the 2.5th percentile of control participants as a
cut-off signifying subclinical acidosis. The second column shows the hazard ratio
reduction per higher SD of the AB_score.

| | Hazard ratios (95% CI) | | | |
|---|---|---|---|---|
| | AB_score | | AB_score | |
| | (SA yes vs. | | (per SD | |
| Cohort | no) | P | higher) | P |
| RENVAS + PUMA | | | | |
| (n = 155) | | | | |
| Unadjusted | 5.7 (2.4 to 13.4) | <0.001 | 0.44 (0.30 to 0.63) | <0.001 |
| Adjusted^{a} | 5.6 (2.2 to 14.1) | <0.001 | 0.46 (0.30 to 0.70) | <0.001 |

^{a}Adjusted for age (years), sex, BMI (kg/m²), baseline eGFR (mL/min/1.73 m²), UACR (mg/g), systolic blood pressure (mmHg), and tCO₂ (mM)

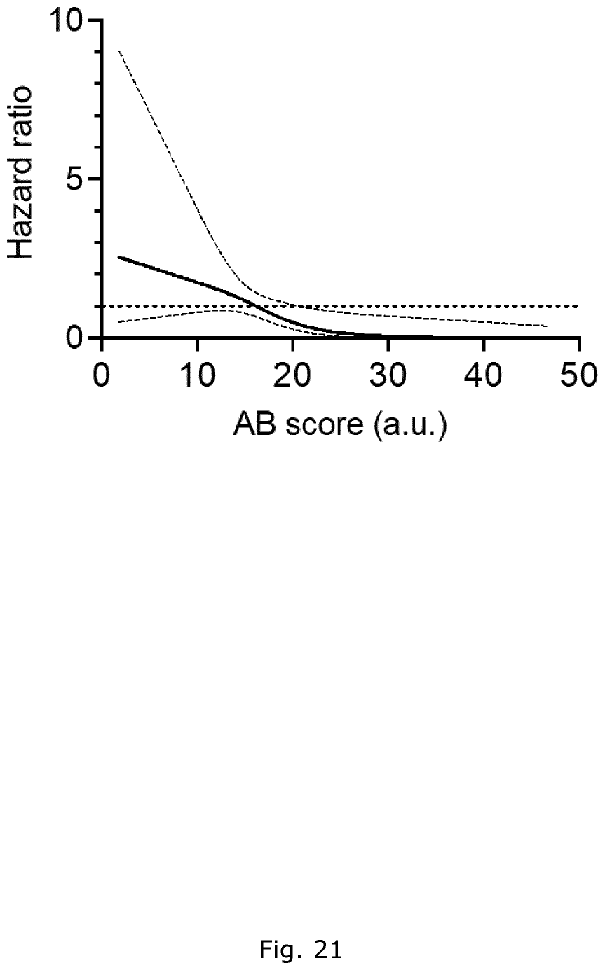

A cubic spline graph of the association between baseline AB_score and CKD progression in the pooled cohort is shown in FIG. 21.

Notably, the present example is based on frozen urine samples stored for up to 10 years before analysis. To indirectly assess acid/base biomarker stability, the inventors compared AB_scores from the older development and validation cohorts with scores obtained from the more recent NNRD. The inventors found no apparent differences in mean AB_score between the older cohorts and the NNRD cohorts suggesting that AB_score is stable during long-term storage.

### Conclusion

Urinary ammonium concentration increases dramatically with decreasing urinary pH in healthy controls, reflecting the normal physiological association between the two parameters. This association is attenuated in patients with CKD (FIG. 1) and a decreased ability to increase urine ammonium concentration as a function of decreasing urine pH results in a low AB_score (FIG. 16B).

In the development cohort (RENVAS), a higher AB_score at baseline was associated with an attenuated mGFR decline after 18 months follow-up. Further, a low AB_score was associated with a markedly increased risk of CKD progression. In the validation cohort (PUMA), these findings were confirmed. Furthermore, a low AB_score or SA status was also associated with a higher risk of a composite outcome of CKD progression or death. Importantly, all these findings persisted after adjustment for known risk factors, such as male sex and baseline albuminuria, eGFR and plasma tCO₂.

Hence, it is found that early acid retention in CKD patients can be recognized in urine samples and that the urine AB_score serves as an independent predictor of CKD progression.

As mentioned before, in current clinical practice, acidosis is diagnosed by measuring blood bicarbonate or plasma tCO₂ while acid retention without evident acidosis is not assessed.

Here, we surprisingly demonstrate that some CKD patients with normal plasma tCO₂ exhibit a urinary phenotype indicative of acid retention, namely low urine pH with concurrent low ammonium, resulting in a low urine AB_score. The key finding from this study is that this phenotype is associated with a markedly increased risk for CKD progression. This increased risk is likely not attributed to differences in bicarbonate levels, i.e., that patients in the lower normal tCO₂ range have increased risk of progression, as the association was still significant after adjusting for tCO₂. Likewise, exclusion of acidotic patients or inclusion of acidosis as a covariable instead of tCO₂ did not impact our findings.

### REFERENCES

Chan J C M, Clin. Biochem. 5, 94-98 (1972).
Elinton J R et al. p. 554-575, American Journal of Medicin, Clinical Studies, October 1960
Schwartz W B et al, “On the mechanism of acidosis in chronic renal disease”, (submitted 1958/accepted Sep. 11, 1958).

1. A method for determining the risk of a subject suffering from chronic kidney disease (CKD) having subclinical acidosis (SA), the method comprising
a) measuring or sensing in a urine sample from a subject, the level of at least two biomarkers selected from the group consisting of
NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻;
b) determining a score based on a relationship between the levels of the at least two biomarkers by comparing to a reference level; and
c) classifying the score as a positive or negative score;
wherein said subject is
at risk of having subclinical acidosis, if said score is negative; and
not at risk of having subclinical acidosis, if said score is positive, wherein, if said subject is at risk of having subclinical acidosis a treatment protocol is initiated.

2. The method according to claim 1, wherein the treatment protocol is selected from the group consisting of acid-reducing dietary regimes, base supplementation, pharmacological treatment, or gastrointestinal proton chelators.

3. (canceled)

4. (canceled)

5. (canceled)

6. The method according to claim 1, wherein the level of NH₄⁺, TA and TB is the excretion of NH₄⁺, TA and TB.

7. The method according to claim 1, wherein the level of NH₄⁺ is the NH₄⁺/creatinine ratio, the level of TA is the TA/creatinine ratio and wherein the level of BB is the BB/creatinine ratio.

8. The method according to claim 1, wherein the level of NH₄⁺ is the NH₄⁺/creatinine ratio and wherein the level of HCO₃⁻ is the HCO₃⁻/creatinine ratio.

9. The method according to claim 1, wherein said relationship is between the two biomarkers NH₄⁺ vs. pH.

10. (canceled)

11. A method for monitoring the development of subclinical acidosis (SA) in a subject suffering from chronic kidney disease (CKD), the method comprising
determining a first score based on a relationship between the levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻, said biomarkers being measured or sensed in a first urine sample from the subject;
determining a second score based on a relationship between the levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻, said biomarkers being measured or sensed in a second urine sample from the subject, wherein the second sample has been obtained at a later time point than the first sample;
comparing corresponding scores in the first and second sample;
wherein
a negative score in the second sample compared to a positive score in the first sample is indicative of development of subclinical acidosis;
a positive score in the second sample compared to positive score in the first sample is indicative of no development of subclinical acidosis;
a negative score in the second sample compared to a negative score in the first sample is indicative of maintained subclinical acidosis;
a positive score in the second sample compared to negative score in the first sample is indicative of improved/lessening of subclinical acidosis.

12. The method according to claim 11, wherein a treatment of subclinical acidosis has taken place between the sampling of the first sample and the second sample.

13. The method according to claim 11, or 12 wherein said a treatment of subclinical acidosis is acid-reducing dietary regimes, base supplementation, pharmacological treatment or gastrointestinal proton chelators.

14. (canceled)

15. (canceled)

16. (canceled)

17. The method according to claim 11, wherein the level of NH₄⁺, TA and TB is the excretion of NH₄⁺, TA and TB.

18. The method according to claim 11, wherein the level of NH₄⁺ is the NH₄⁺/creatinine ratio, the level of TA is the TA/creatinine ratio and wherein the level of BB is the BB/creatinine ratio.

19. The method according to claim 11, wherein the level of NH₄⁺ is the NH₄⁺/creatinine ratio and wherein the level of HCO₃⁻ is the HCO₃⁻/creatinine ratio.

20. The method according to claim 11, wherein said relationship is between said two biomarkers NH₄⁺ vs. pH in said first urine sample and said second urine sample.

21. (canceled)

22. (canceled)

23. A method for determining the effect of a treatment protocol against subclinical acidosis (SA) for a subject suffering from chronic kidney disease (CKD), the method comprising
determining a first score based on a relationship between the levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻, said biomarkers being measured or sensed in a first urine sample from the subject;
determining a second score based on a relationship between the levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻, said biomarkers being measured or sensed in a second urine sample from the subject, wherein the second sample has been obtained at a later time point than the first sample;
wherein the treatment protocol has been initiated or completed before the sampling of the first sample or initiated, continued or completed between the sampling of the first and second sample,
comparing scores of the first sample and the second sample; wherein
a negative score in the second sample compared to a positive score in the first sample is indicative of the treatment protocol not being effective against subclinical acidosis;
a negative score in the second sample compared to a negative score in the first sample is indicative of the treatment protocol not being effective against subclinical acidosis;
a positive score in the second sample compared to negative score in the first sample is indicative of the treatment protocol being effective against subclinical acidosis.

24. The method according to claim 23, wherein the treatment is acid-reducing dietary regimes, base supplementation, pharmacological treatment or gastrointestinal proton chelators.

25. (canceled)

26. (canceled)

27. (canceled)

28. (canceled)

29. The method according to claim 23, wherein the level of NH₄⁺ is the NH₄⁺/creatinine ratio, the level of TA is the TA/creatinine ratio and wherein the level of BB is the BB/creatinine ratio.

30. The method according to claim 23, wherein the level of NH₄⁺ is the NH₄⁺/creatinine ratio and wherein the level of HCO₃⁻ is the HCO₃⁻/creatinine ratio.

31. The method according to claim 23, wherein said relationship is between said two biomarkers NH₄⁺ vs. pH in said first urine sample and said second urine sample.

32. (canceled)

33. (canceled)

34. Use of urine sample levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻ as biomarkers for determining the risk of a subject of having subclinical acidosis.

35. Use of urine sample levels of at least two biomarkers selected from the group consisting of NH₄⁺ vs. pH, NH₄⁺ vs. titratable acid (TA), NH₄⁺ vs. total base (TB), NH₄⁺ vs. base buffers (BB), and/or NH₄⁺ vs. HCO₃⁻ as biomarkers for determining the risk of a subject of having subclinical acidosis to predict serious clinical event-free survival of said subject.
